(19) United States
(12) Patent Application Publication
(10) Pub. No.: US 2004/0086504 A1
(43) Pub. Date: May 6, 2004

(54) Cyr61 as a target for treatment and diagnosis of breast cancer

(76) Inventors: Deepak Sampath, Rosemont, PA (US); Zhiming Zhang, Malvern, PA (US); Richard Winneker, Penllyn, PA (US)

Correspondence Address: Paul F Fehlner, Darby & Darby, Post Office Box 5257, New York, NY, 10150-5257, US

(21) Appl. No.: 10/312,459

(22) PCT Filed: Jun. 21, 2001
(86) PCT No.: PCT/US01/19823

(51) Int. Cl. C07H 21/04; A61K 39/395; C12P 21/02; C12N 5/06; C07K 16/30

(52) U.S. Cl. 424/143.1; 530/388.22; 435/69.1; 435/320.1; 435/334; 514/44; 536/23.53

(57) ABSTRACT

The present invention contemplates methods of preventing or inhibiting breast cancer cell proliferation, compounds and compositions that interfere with or block sex steroid or growth factor binding to and induction of the Cyr61 gene and methods of screening ligands that regulate Cyr61 protein expression. The present invention further contemplates compounds that block Cyr61 activity. The invention further relates to methods of diagnosing and staging patients with cancers associated with an upregulation of Cyr61 expression. Assay methods and kits are also disclosed.

### PRIORITY

The present application claims priority from U.S. Provisional Patent Application Serial No. 60/213,182, filed Jun. 21, 200, and U.S. Provisional Patent Application Serial No. 60/291,510, filed May 16, 2001, which are hereby incorporated by reference in their entireties.

### FIELD OF THE INVENTION

The present invention relates to methods of preventing or inhibiting breast cancer cell proliferation, compounds and compositions that interfere with or block sex steroid or growth factor binding to and induction of the Cyr61 gene, and compounds that block Cyr61 activity. The present invention also relates to methods of screening ligands that regulate Cyr61 protein expression. The invention further relates to methods of diagnosing and staging patients with cancers associated with an upregulation of Cyr61 expression. The invention also describes oligonucleotides, antisense constructs, antibodies, neutralizing antibodies, and pharmaceutical compositions related thereto. Transgenic animals are also contemplated by the present invention.

### BACKGROUND OF THE INVENTION

Breast cancer is the leading cause of cancer death among non-smoking women today (Adami, et al., Sem. In Cancer Biol., 1998, 8:255). Although a number of genetic and environmental factors have been implicated in the development of mammary epithelial neoplasia, tumorigenesis appears to be under hormonal regulation.

An emerging group of growth factor-regulated immediate-early genes that play a role in development, cell proliferation, and tumorigenesis belongs to the CCN (CTGF/Cyr61/Cef10/NOVH) family. All CCN proteins (1) display a high degree of conservation among family members and across species; (2) are cysteine-rich and structurally similar to extracellular matrix-associated molecules; (3) are composed of multifunctional modular domains; and (4) have been shown to mediate a variety of cell functions such as cell adhesion, cell migration, mitogenesis, cell survival, and differentiation (Law and Lam, Experimental Cell Res, 1999, 248:44).

Cyr61 is a secreted, cysteine-rich heparin-binding protein that associates with the cell surface and the extracellular matrix. Specifically, Cyr61 has been shown to be involved in developmentally regulated processes including, angiogenesis and chondrogenesis. The Cyr61 protein possesses many biochemical features that resemble the Wnt-1 protein and other growth factors (Yang and Law, Cell Growth & Diff, 1991, 2:351). The human Cyr61 protein is 381 amino acids in length with a molecular mass of about 42 kilo-daltons (kDa). See FIG. 1 and PCT Application No. WO 97/339950. The Cyr61 gene is localized in the short arm of chromosome 1 (1p22-31) (Charles et al., Oncogene, 1991, 8:23; Jay et al., Oncogene, 1997, 14:1753), and the gene was identified by differential hybridization screening of a cDNA library of serum-stimulated BALB/c3T3 fibroblasts (See FIG. 2 and Law and Nathans, P.N.A.S., 1987, 84:1182). Comparison of the human and murine Cyr61 sequences indicates that they are 91% similar (PCT application No. WO 97/339950).

The present inventors have found that regulation of Cyr61 expression and activities is useful in the prevention, diagnosis, and treatment of breast cancer.

### SUMMARY OF THE INVENTION

The present invention provides methods for preventing or inhibiting breast cancer cell proliferation. These methods comprise administering to a subject, a Cyr61 neutralizing antibody which blocks activities associated with the proliferation and/or growth of breast cancer cells. The neutralizing antibody is used in effective amounts sufficient to block or reduce Cyr61 activities associated with breast cancer cell proliferation and/or growth. In one embodiment, the antibody is conjugated with an anti-tumor agent. In one embodiment, the neutralizing antibody blocks sex steroid induced synthesis of Cyr61 DNA and proliferation of breast cancer cells. In an alternative embodiment, the neutralizing antibody blocks growth factor induced synthesis of Cyr61 DNA and proliferation of breast cancer cells, where the growth factor is selected from the group consisting of epidermal growth factor, heparin binding epidermal growth factor, and basic fibroblastic growth factor.

Further contemplated are methods for preventing or inhibiting breast cancer cell proliferation which comprise administering to a subject an amount of a compound effective to inhibit the interaction of a sex steroid response element of the Cyr61 promoter and a sex steroid receptor associated with the Cyr61 promoter, to block a sex steroid receptor which regulates the Cyr61 promoter, to inhibit the synthesis of DNA or mRNA encoding Cyr61, to inhibit the upregulation of the expression of Cyr61, or to inhibit the binding of Cyr61 to cognate receptor(s) or interacting protein(s). In a specific embodiment, the sex steroid is an estrogenic compound, progestational compound, or androgenic compound. In another embodiment, the sex steroid response element is an estrogen response element or a progesterone/androgen response element. In another embodiment, the expression of Cyr61 is upregulated by a growth factor such as, but not limited to, epidermal growth factor, heparin binding epidermal growth factor, and basic fibroblastic growth factor.

The present invention also provides antisense constructs such as an oligonucleotide which binds under high stringency conditions to DNA or mRNA encoding Cyr61, a vector comprising such oligonucleotides, and pharmaceutical compositions comprising a therapeutically effective amount of such oligonucleotides or vectors. In one embodiment, the oligonucleotide is non-naturally occurring.

Also provided are antibodies which neutralize Cyr61 activity and pharmaceutical compositions containing therapeutically effective amounts of these antibodies. These antibodies may be polyclonal or monoclonal, chimeric, humanly acceptable and conjugated to an anti-tumor agent or antibiotic such as, for example, calicheamicin. Special mention is made of antibodies which bind to amino acids 163-229 of SEQ ID NO: 2 (see FIG. 1) and antibodies which bind to amino acids 371-381 of SEQ ID NO: 2 (see FIG. 1). Antibodies that bind to one or more ligands of a sex steroid receptor, such as, but not limited to the estrogen receptor, progesterone receptor, and androgen receptor, which regulates the promoter of the gene which encodes Cyr61, also are contemplated. An antibody which binds to an epitope of Cyr61 is also disclosed. Pharmaceutical compositions comprising an antibody also are contemplated.

The present invention provides for compounds that inhibit the interaction of a sex steroid response element of Cyr61 gene and a sex steroid receptor. The steroid response element resides within the Cyr61 promoter.

The present invention also provides for antibodies that may bind to one or more ligands of a sex steroid receptor which regulates the promoter gene that encodes Cyr61. Pharmaceutical composition containing therapeutically effective amounts of these antibodies are also contemplated.

Also provided are methods for diagnosing or staging breast cancer. These methods comprise determining the level of Cyr61 in a breast cell that is obtained from breast tissue suspected of being positive for breast cancer and comparing that level to the level of Cyr61 in normal breast tissue. An increase in the level of Cyr61 in the suspect tissue over the level of Cyr61 in the normal tissue indicates the presence of breast cancer in the suspect tissue. The level of Cyr61 in this method can be determined by exposing the suspect and the normal tissues to an antibody as described above and then comparing the amount of antibody bound by each tissue. An increase in the level of antibody bound by the suspect tissue over the level of antibody bound by the normal tissue indicates the presence of breast cancer in the suspect tissue.

Other methods for diagnosing or staging breast cancer comprises determining whether breast tissue suspected of being positive for breast cancer is (i) ER/Cyr61 positive, (ii) PR/Cyr61 positive (iii) ER/PR/Cyr61 positive, (iv) AR/Cyr61 positive, or (v) PR/EGFR/Cyr61 positive. The presence of (i), (ii), (iii), (iv), or (v) indicates a likelihood that said suspect tissue is cancerous and the aforementioned results can be utilized to design specific treatment regimens.

Methods of screening for a compound which inhibits or prevents breast cancer cell proliferation are also provided. These methods comprise determining a first amount of Cyr61 expressed by breast cancer cells exposed to the compound, where the breast cancer cell overexpresses Cyr61; and comparing the first amount of Cyr61 to a second amount of Cyr61 expressed by the breast cancer cells that has not been exposed to the compound. If the first amount is less than the second amount, this is an indication that the compound may inhibit or prevent breast cancer cell proliferation.

Other methods of screening for a compound which inhibits or prevents breast cancer cell proliferation involve determining whether the compound inhibits the interaction of a sex steroid response element of the Cyr61 promoter, the compound binds with a sex steroid receptor which regulates the Cyr61 promoter, or the compound blocks interaction with Cyr61 receptors or interacting proteins.

Additionally, the present invention provides a transgenic non-human animals comprising DNA, such as, for example, human DNA which can be induced to overexpress Cyr61 in breast tissue.

Kits for diagnosing or staging breast cancer are also provided. These kits include antibodies or oligonucleotides as described above.

Methods for screening for compounds that regulate Cyr61 mRNA transcription are also provided. Transcription may be regulated by a receptor or a non-receptor mediated mechanism. These methods include detecting a difference in the level of Cyr61 mRNA in a population of cells sufficient to transcribe a detectable amount of mRNA encoding Cyr61.

Assay systems for detecting the presence of breast cancer are also provided in which the level of Cyr61 polynucleotide isolated from breast cancer tissue is detected. An upregulation of Cyr61 mRNA compared to normal mammary tissue indicates the presence of breast cancer. In an alternate embodiment, the level of Cyr61 protein isolated from breast cancer tissue, is detected and an upregulation of Cyr61 protein compared to normal mammary tissue indicates the presence of breast cancer.

### BRIEF DESCRIPTION OF THE DRAWINGS

FIGS. 1(a-b). An illustration of the amino acid sequence of the human Cyr61 protein (SEQ ID NO: 2).

FIGS. 2(a-c). An illustration of the cDNA (SEQ ID NO: 1) encoding the human Cyr61 protein.

FIGS. 3(a-b). An illustration of a Northern blot of total RNA from T47D and MCF-7 adenocarcinoma cells demonstrating the regulation of Cyr61 mRNA transcription by R5020 and 17β-estradiol, respectively.

FIG. 4. An illustration of a Northern blot of total RNA from T47D adenocarcinoma cells demonstrating the effects of transcription inhibitor actinomycin D and protein synthesis inhibitor cycloheximide on progestin induced regulation of Cyr61 transcription.

FIG. 5. An illustration of a Northern blot of total RNA from MCF-7 adenocarcinoma cells demonstrating the effects of transcription inhibitor actinomycin D and protein synthesis inhibitor cycloheximide on estrogen induced regulation of Cyr61 transcription.

FIG. 6. An illustration of a Western blot of proteins from T47D adenocarcinoma cells demonstrating the regulation of Cyr61 protein expression by R5020.

FIG. 7. A graph of the time course of mRNA induction in T47D and MCF-7 cells after treatment with R5020 and 17β-estradiol, respectively.

FIGS. 8(a-b). An illustration of a Western blot of proteins from T47D and MCF-7 adenocarcinoma cells demonstrating the upregulation of Cyr61 protein levels.

FIGS. 9(a-c). (a-b) Illustrations of Western blots from ER+/PR+/EGFR+ and ER−/PR−/EGFR+ breast cancer tissues probed for the upregulation of Cyr61. (c) A bar graph showing the level of Cyr61 proteins in ER+/PR+/EGFR+ and ER−/PR−/EGFR+ breast cancer cells compared to normal mammary tissue.

FIGS. 10(a-f). An illustration of in situ hybridization studies that indicate the localization of Cyr61 mRNA in breast cancer cells.

FIGS. 11(a-d). A bar graph comparing a test compound to the total number of cells showing of the effects of Cyr61 neutralizing antibodies on estrogen and epidermal growth factor induced cell proliferation and DNA-synthesis in MCF-7 cells.

FIGS. 12(a-b). A bar graph comparing a test compound to the total number of cells showing the effects of Cyr61 neutralizing antibodies on progestin and serum induced DNA synthesis in T47D cells.

FIGS. 13(a-b). A bar graph comparing a test compound to DNA synthesis showing the effects of Cyr61 neutralizing antibodies on EGF and HB-EGF stimulation of DNA synthesis in breast cancer cells.

FIGS. 14(a-e). (a and c) An illustration of Northern blots demonstrating the effects of EGF in T47D and MCF-7 cells, respectively. (b and d) An illustration of Northern blots demonstrating the effects of EGF and R5020 in T47D cells (b) or 17β-estradiol and EGF treated MCF-7 cells (d). (e) A graph demonstrating the time course of mRNA transcription in cells treated with EGF, EGF and R5020, and EGF and 17β-estradiol.

FIGS. 15(a-d). Illustrations of Northern blots demonstrating the effects of DHT in AR+ MDA-MB-453 (a and b) and ZR-75-1 (c and d) cells. Kinetics of Cyr61 mRNA induction in MDA-MB-453 (a) and ZR-75-1 (c) cells was evaluated after treatment with 1.0 nM DHT at specified time points. Determination of the DHT EC50 for Cyr61 upregulation in cells was evaluated at specified concentrations.

FIGS. 16(a-d). (a) Illustration of Western blots demonstrating the time-dependent upregulation of Cyr61 protein in MDA-MB-453 cells after 0, 0.5, 2.0, 4.0, 8.0 and 24.0 h treatment with 1 nM DHT. (c) Illustration of Western blots demonstrating the dose-dependent upregulation of Cyr61 protein in MDA-MB-453 cells at 0, 0.1, 1.0, 10.0, and 100.0 nM DHT for 0.5 h. (b and d) Bar graphs illustrating Cyr61 protein levels. Numerical values are based on the relative optical density (OD) of the band size and the total amount of Cyr61 was normalized to the level of cytokeratin. The fold expression of Cyr61 was calculated by dividing the ratio of Cyr61/cytokeratin in treated cells by untreated cells. Significant increase in levels compared to untreated controls, p<0.0001.

FIGS. 17(a-b). (a) Illustration of Northern blot of total RNA isolated from MDAMB-453 cells following treatments with 1.0 nM DHT, 10 μg/ml cyclohexamide (Chx.), 1 μg/ml actinomycin D (Act. D) or 100 nM 2-OH Flu for 0.5 h. (b) A bar graph illustrating the fold expression of Cyr61 mRNA (as calculated by dividing of Cyr61/GAPDH)* Significant increase in levels compared to untreated controls, p<0.0001.

FIGS. 18(a-b). Illustration of Northern blot of total RNA isolated from MDA-MB-231 cells following treatments with 1.0 nM DHT (a) or 20 ng/ml EGF (b) at specified time points.

FIGS. 19(a-c). (a-b) Illustration of Western blot analysis of breast tumors (T) and autologous normal mammary controls (N) tissue protein extracts of 4 representative patients (#1-4) that were AR− (a) and 4 representative patients (# 5-8) that were AR+ (b). (c) Bar graph representing Cyr61 increase.

FIGS. 20(a-b). Bar graphs illustrating the effects of anti-Cyr61 neutralizing antibodies on DHT and EGF-dependent DNA synthesis in MDA-MB-453 cells.* p<0.0001.

FIGS. 21(a-b). Bar graphs illustrating the effects of anti-Cyr61 neutralizing antibodies on DHT and EGF-dependent proliferation in MDA-MB-453 cells.* p<0.0001.

FIGS. 22(a-d). Line graphs illustrating the effects of anti-Cyr61 neutralizing antibodies on R5020 and EGF-dependent DNA cell growth in T47D cells. Cell proliferation experiments were performed in quadruplicates and repeated three times. Numerical values represent total cell numbers+SEM. *p<0.0001.

FIGS. 23(a-b). Illustration of Western blot of stage II invasive ductal breast tumors (T) and autologous normal mammary controls (N) tissue protein extracts generated from 5 patients (#1-5) that were PR−/EGFR+ and 5 patients (# 6-10) that were PR+/EGFR+.

### DETAILED DESCRIPTION OF THE INVENTION

The present invention describes methods of preventing or inhibiting breast cancer cell proliferation, diagnosing or staging breast cancer, and screening for compounds which inhibit or prevent breast cancer cell proliferation. These methods evaluate sex steroid and growth factor mediated regulation of Cyr61 transcription and translation and levels in samples of interest. The present invention also advantageously provides for screening assays and kits. The assay system of the invention is suitable for high throughput screening, e.g., screening thousands of compounds per assay.

The present invention also provides Cyr61 nucleic acids, including oligonucleotide primers, probes, and antisense constructs, and Cyr61 regulatory sequences; Cyr61-specific antibodies; and related methods of using these materials to detect the presence of Cyr61 proteins or nucleic acids and in screens for agonists and antagonists of Cyr61 for breast cancer. The invention also describes pharmaceutical compositions of these materials.

### General Definitions

The term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. Purity can be evaluated by chromatography, gel electrophoresis, immunoassay, composition analysis, biological assay, and other methods known in the art.

A “sample” refers to a biological material which can be tested for the presence of Cyr61 protein or Cyr61 nucleic acids. Such samples can be obtained from subjects, such as humans and non-human animals, and include tissue, especially mammary glands, biopsies, blood and blood products; plural effusions; cerebrospinal fluid (CSF); ascites fluid; and cell culture.

The term “non-human animals” includes, without limitation, laboratory animals such as mice, rats, rabbits, hamsters, guinea pigs, etc.; domestic animals such as dogs and cats; and, farm animals such as sheep, goats, pigs, horses, and cows.

The term “ability to elicit a response” refers to the ability of a ligand to agonize or antagonize receptor activity.

The term “transformed cell” refers to a modified host cell that expresses a functional protein expressed from a vector encoding the protein of interest. Any cell can be used, but preferred cells are mammalian cells.

The term “assay system” is one or more collections of such cells, e.g., in a microwell plate or some other culture system. To permit evaluation of the effects of a test compound on the cells, the number of cells in a single assay system is sufficient to express a detectable amount of the regulated Cyr61 mRNA and protein expression. The methods of the invention are suitable cells of the invention that are particularly suitable for an assay system for test ligands that modulate transcription and translation of the Cyr61 gene.

A “test compound” is any molecule, such as, for example, a sex steroid that can be tested for its ability to modulate Cyr61 expression and/or activity.

The terms “cancer” or “tumors” refers to group of cells that display uncontrolled division. In a specific embodiment, the cancer is breast cancer and particularly infiltrating ductal carcinomas. The term “cell proliferation” refers to the growth of a cell or group of cells.

The term “humanly acceptable” refers to compounds or antibodies that are modified so as to be useful in treatment of human diseases or disorders. In a specific embodiment, antibodies (polyclonal or monoclonal) are modified so that they are humanly acceptable. In one embodiment, this requires the antibodies to be humanized or primatized.

The use of italics generally indicates a nucleic acid molecule (e.g., Cyr61 cDNA, gene, etc.); normal text generally indicates the polypeptide or protein. Alternatively, whether a nucleic acid molecule or a protein is indicated can be determined by the content.

The term “amplification” of DNA refers to the use of polymerase chain reaction (PCR) to increase the concentration of a particular DNA sequence within a mixture of DNA sequences. For a description of PCR see Saiki et al., Science, 239:487, 1988.

The term “sequence-specific oligonucleotides” refers to related sets of oligonucleotides that can be used to detect allelic variations or mutations in the Cyr61 gene.

The term “nucleic acid molecule” refers to the phosphate ester form of ribonucleosides (RNA molecules) or deoxyribonucleosides (DNA molecules), or any phosphoester analogs, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

The terms “polynucleotide” or “nucleotide sequence” is a series of nucleotide bases (also called “nucleotides”) in DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and antisense polynucleotide. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thiouracil, thio-guanine and fluoro-uracil.

The polynucleotides may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme. Host cells can further be used for screening or other assays, as described infra.

Generally, a DNA sequence having instructions for a particular protein or enzyme is “transcribed” into a corresponding sequence of RNA. The RNA sequence in turn is “translated” into the sequence of amino acids which form the protein or enzyme. An “amino acid sequence” is any chain of two or more amino acids. Each amino acid is represented in DNA or RNA by one or more triplets of nucleotides. Each triplet forms a codon, corresponding to an amino acid. The genetic code has some redundancy, also called degeneracy, meaning that most amino acids have more than one corresponding codon.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme.

The term “gene”, also called a “structural gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription.

A “promoter sequence” is a DNA regulatory region capable of binding a secondary molecule which in a cell and initiating transcription of a coding sequence.

A coding sequence is “under the control” or “operatively associated with” of transcriptional and translational control sequences in a cell when RNA polymerase transcribes the coding sequence into mRNA, which is then trans-RNA spliced (if it contains introns) and translated into the protein encoded by the coding sequence.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “transfection” means the introduction of a foreign nucleic acid into a cell. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include plasmids, phages, viruses, etc.

A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.”

The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is a such an element operatively associated with a different gene than the one it is operatively associated with in nature.

The terms “mutant” and “mutation” mean any detectable change in genetic material, e.g. DNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence. The term “variant” may also be used to indicate a modified or altered gene, DNA sequence, enzyme, cell, etc., i.e., any kind of mutant.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 0.25% milk, and no formamide; or 30% formamide, 5×SSC, 0.5% SDS. Moderate stringency hybridization conditions correspond to a higher Tₘ, e.g., 40% formamide, with 5× or 6×SCC. High stringency hybridization conditions correspond to the highest Tm, e.g., 50% formamide, 5× or 6×SCC. SCC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

The term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of Cyr61, or to detect the presence of nucleic acids encoding Cyr61. Generally, oligonucleotides are prepared synthetically, preferably on a nucleic acid synthesizer.

### Cancers

As mentioned above, the term “cancer” refers to cells that display uncontrolled proliferation or division. The degree to which a cancer has spread beyond its original location is referred to as the “stage” of the cancer. Lower stages, such as stages I and II, are generally more confined to their site or region of origin than advanced stages (III and IV). See, e.g., The Merck Manual, 15^{th} Edition, Merck, Sharp, & Dohme Research Laboratories (1987).

Breast cancers refer to a class of cancers that are associated with development in the breast of women and men. The most common type of breast cancer is invasive ductal carcinoma. It occurs most frequently in women in their 50's and appears to spread from the breast into the lymph nodes. Estrogen receptors (ER), progesterone receptors (PR), and androgen receptors (AR) are molecules within many breast cancer cells. The cancer along with the increased levels of Cyr61 in breast cancer cells along with the presence or absence of ER, PR, or AR have prognostic and predictive value and can be used as a basis for designing treatment regimens. Presence of these receptor molecules within the cancer cells is referred to as estrogen receptors positive (ER+), progesterone receptors positive (PR+), and/or androgen positive (AR+) tumors, while absence is referred to as estrogen receptors negative (ER−), progesterone receptors negative (PR-) and/or androgen receptors negative (AR−).

### Antibodies and Antisense Constructs

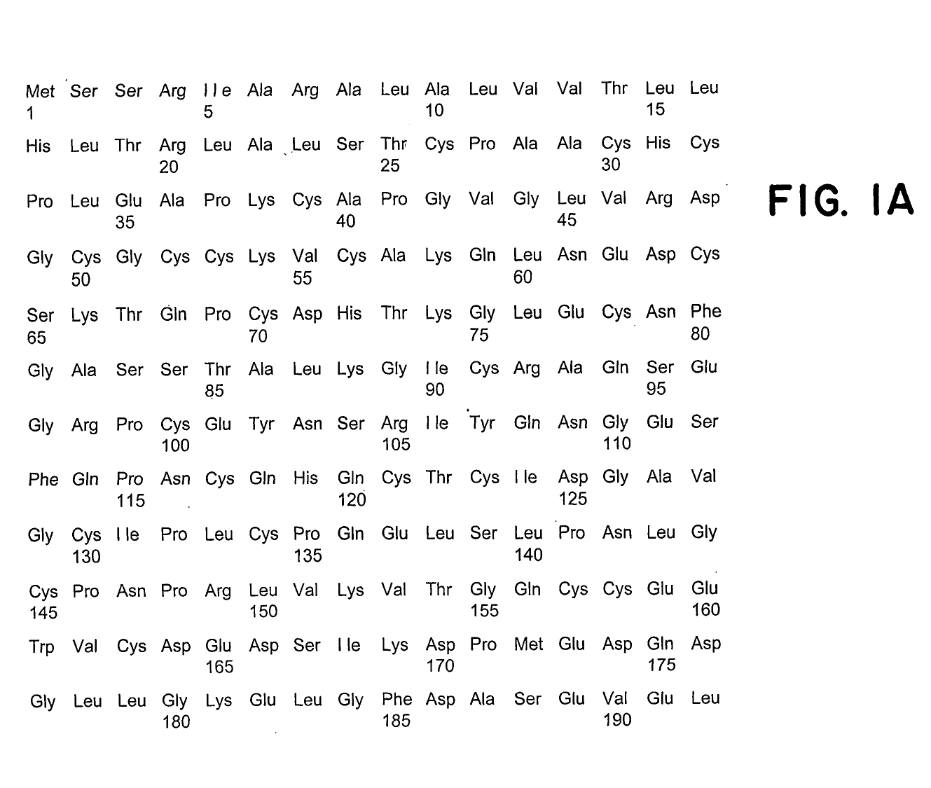

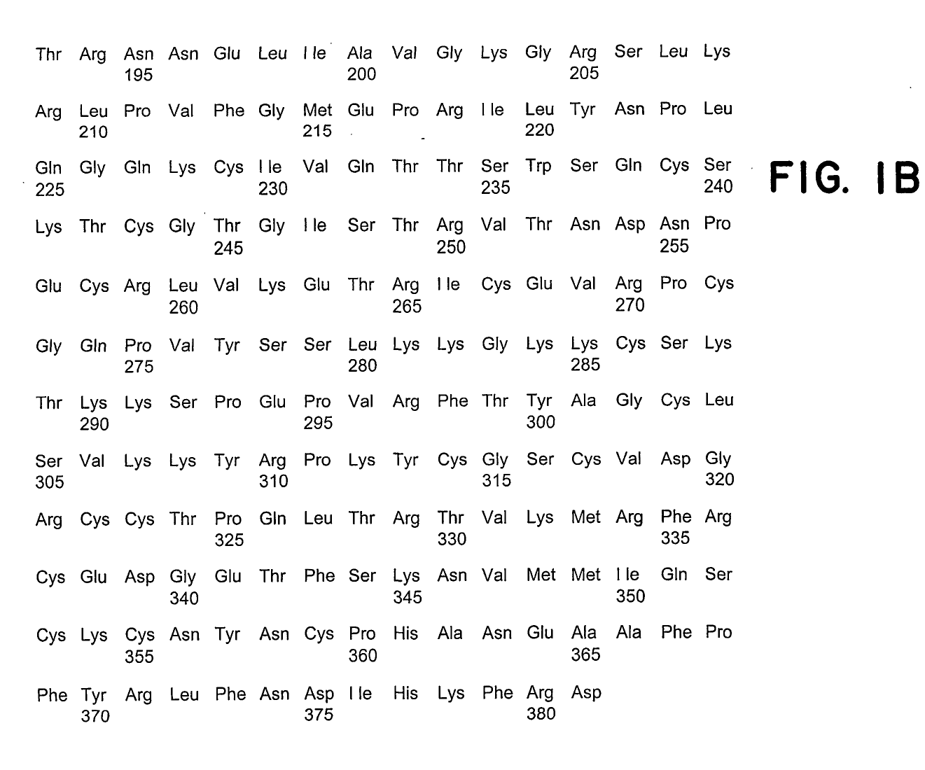

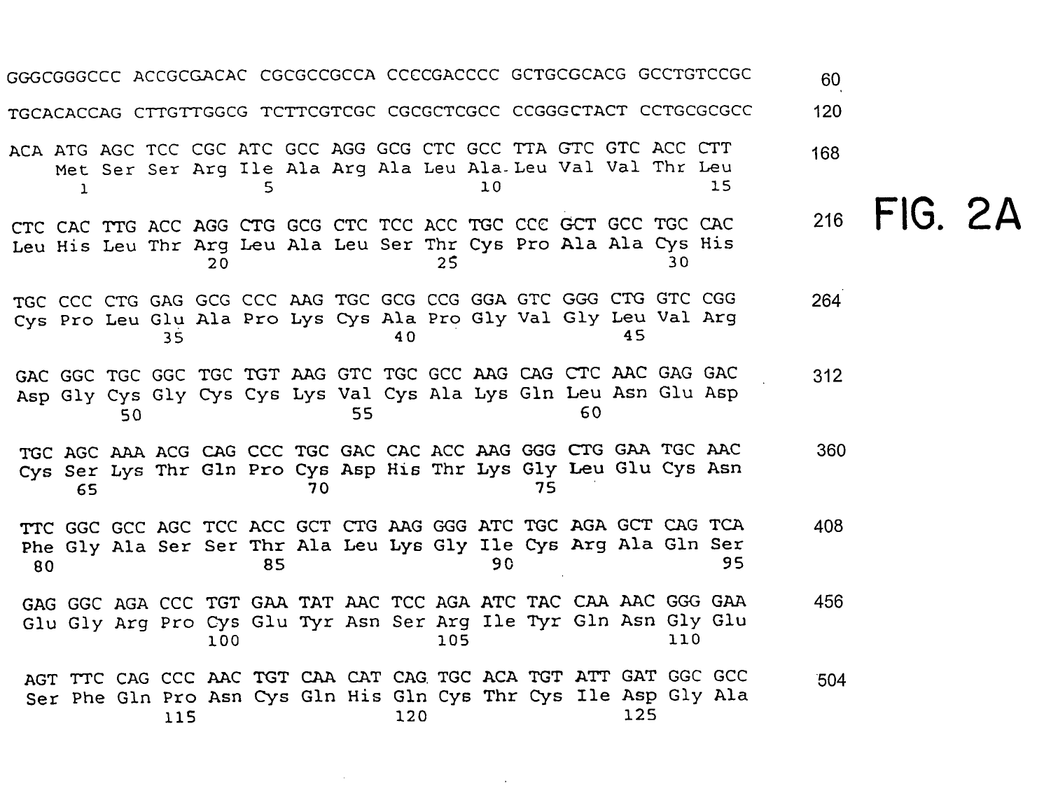

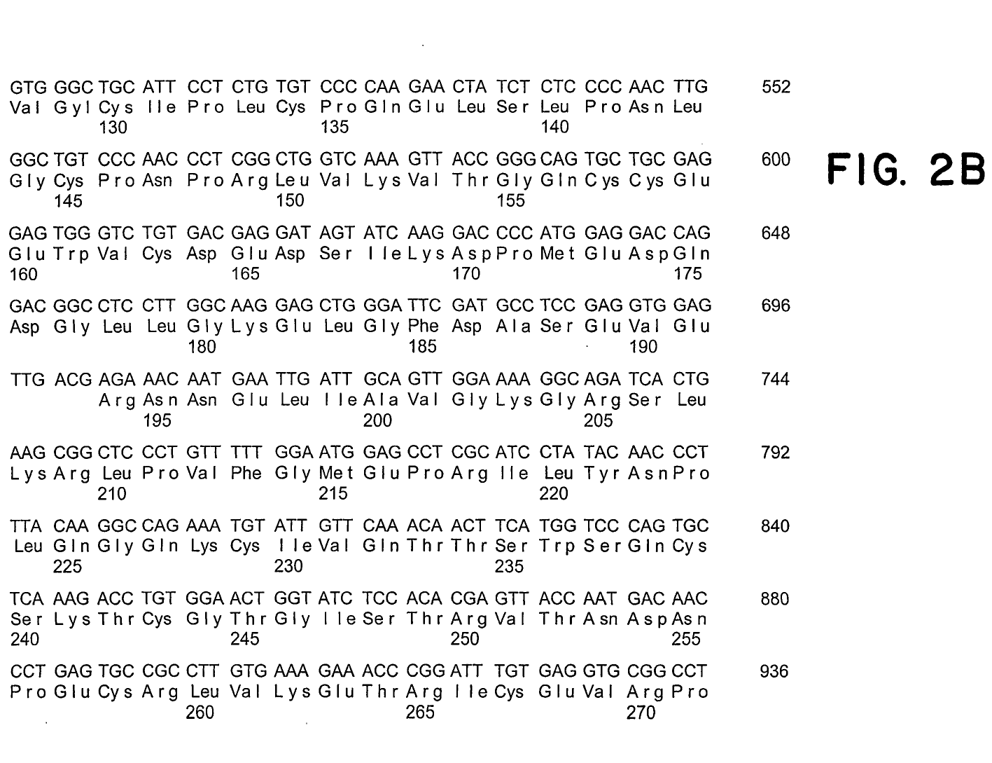

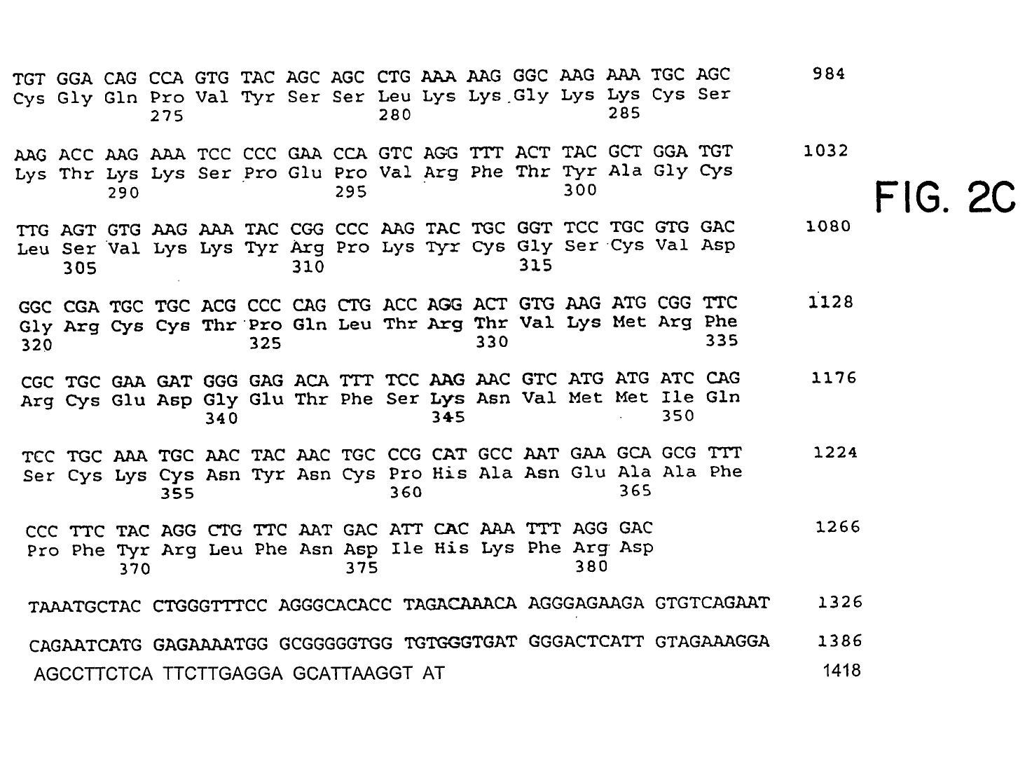

The present invention describes neutralizing antibodies that may be used to block the activity of Cyr61 in cells and specifically in cancer cells such as breast cancers. According to the invention, Cyr61 polypeptides produced recombinantly or by chemical synthesis, and fragments or other derivatives, may be used as an immunogen to generate antibodies that recognize the Cyr61 polypeptide or portions thereof. Such antibodies include, but are not limited to, polyclonal, monoclonal, humanized, primatized, chimeric, single chain, Fab fragments, and an Fab expression library. An antibody that is specific for human Cyr61 may recognize a wild-type or mutant form of Cyr61. Preferred neutralizing antibodies are produced to, but not limited to, the amino acids 163-229 and 371-381 in SEQ ID NO. 2 (See FIG. 1).

The invention also describes pharmaceutical compositions that modulate the transcription of the Cyr61 gene by sex steroid receptors (ovarian and testicular) and growth factors. These receptors recognize consensus sex steroid responsive elements (ERE for estrogen receptors, PRE/ARE for progesterone receptors and androgen receptors) in the promoter region of the Cyr61 gene. In addition to the DNA binding region, the steroid receptors also contain at least two regions that initiate gene transcription. The estrogen receptor recognizes an estrogen response element (ERE) having the DNA sequence including, but not limited to, 5′-GGTCAxxxTGACC-3′ (SEQ ID NO:3) and the progesterone receptor and androgen receptor recognize a progesterone receptor/androgen receptor element (PRE/ARE) having the DNA sequence including, but not limited to, 5′TGTACAxxxTGTTCT-3′ (SEQ ID NO:4); where x represents any nucleotide.

Pharmaceutical compositions that prevent ER, PR, and AR binding to the Cyr61 promoter are contemplated in this invention. Antibodies to the amino acid sequences of the sex steroid receptors that recognize the specific consensus sequences are contemplated in this invention. As mentioned above, polypeptides produced recombinantly or by chemical synthesis, and fragments or other derivatives, may be used as an immunogen to generate antibodies that recognize the steroid receptor polypeptide regions that comprise the gene binding sequence. Polyclonal, monoclonal, humanized, primatized, chimeric, single chain, Fab fragments, and an Fab expression library antibodies are included.

Various procedures known in the art may be used for the production of polyclonal antibodies to polypeptides, derivatives, or analogs. For the production of antibody, various host animals, including but not limited to rabbits, mice, rats, sheep, goats, etc, can be immunized by injection with the polypeptide or a derivative (e.g., fragment or fusion protein). The polypeptide or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, KLH, dinitrophenol, and potentially useful human adjuvants such as BCG (bacille Calmette-Guerin) and Corynebacterium parvum.

Monoclonal antibodies directed toward a Cyr61 polypeptide, fragment, analog, or derivative thereof, may be prepared by any technique that provides for the production of antibody molecules by continuous cell lines in culture may be used. These include but are not limited to the hybridoma technique originally developed by Kohler and Milstein Nature 256:495-497, 1975), as well as the trioma technique, the human B-cell hybridoma technique (Kozbor et al., Immunology Today 4:72, 1983; Cote et al., Proc. Natl. Acad. Sci. U.S.A. 80:2026-2030, 1983), and the EBV-hybridoma technique to produce human monoclonal antibodies (Cole et al., in Monoclonal Antibodies and Cancer Therapy, Alan R. Liss, Inc., pp. 77-96, 1985). “Chimeric antibodies” may be produced (Morrison et al., J. Bacteriol. 159:870, 1984; Neuberger et al., Nature 312:604-608, 1984; Takeda et al., Nature 314:452-454, 1985) by splicing the genes from a non-human antibody molecule specific for a polypeptide together with genes from a human antibody molecule of appropriate biological activity.

In the production and use of antibodies, screening for or testing with the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc.

The foregoing antibodies can be used in methods known in the art relating to the localization and activity of the polypeptide, e.g., for Western blotting, imaging the polypeptide in situ, measuring levels thereof in appropriate physiological samples, etc. using any of the detection techniques mentioned above or known in the art. Such antibodies can also be used in assays for ligand binding, e.g., as described in U.S. Pat. No. 5,679,582. Antibody binding generally occurs most readily under physiological conditions, e.g., pH of between about 7 and 8, and physiological ionic strength. The presence of a carrier protein in the buffer solutions stabilizes the assays. While there is some tolerance of perturbation of optimal conditions, e.g., increasing or decreasing ionic strength, temperature, or pH, or adding detergents or chaotropic salts, such perturbations will decrease binding stability.

In a specific embodiment, antibodies that agonize or antagonize the activity of Cyr61 polypeptide can be generated. In particular, intracellular single chain Fv antibodies can be used to regulate (inhibit) Cyr61. Such antibodies can be tested using the assays described below for identifying ligands.

In another specific embodiment, antibodies of the present invention are conjugated to a secondary component, such as, for example, a small molecule, polypeptide, or polynucleotide. The conjugation may be produced through a chemical modification of the antibody, which conjugates the antibody to the secondary component. The conjugated antibody will allow for targeting of the secondary component, such as, for example, an anti-tumor agent to the site of interest. The secondary component may be of any size or length. Examples of anti-tumor agents include, but are not limited to, chemotherapeutic agents, toxins, radioactive isotopes, mitotic inhibitors, cell-cycle regulators, and anti-microtubule disassembly compounds. The secondary component may be an antibiotic including, but not limited to, calicheamicin. An example of an anti-microtubule disassembly compounds is taxol (Wani et al., J. Amer. Chem. Soc., 1971, 93:2325-2327; Horwitz,et al., Nature, 1979, 277:665).

A further aspect of this invention relates to the use of antibodies, as discussed supra, for targeting a pharmaceutical compound. In this embodiment, antibodies against Cyr61 are used to present specific compounds to cancerous cells. The compounds, preferably an anti-tunor agent or an anti-cancer agent, when conjugated to the antibodies are referred to as targeted compounds or targeted agents. Methods for generating such target compounds and agents are known in the art. Exemplary publications on target compounds and their preparation are set forth in U.S. Pat. Nos. 5,053,934; 5,773,001; and 6,015,562.

Any desired agent (known as an anti-tumor agent) having activity against cancer cells may be employed in generating the targeted agent. Examples of such compounds are discussed in U.S. Pat. No. 6,015,562. See specifically U.S. Pat. Nos. 4,971,198; 5,079,233; 4,539,203; 4,554,162; 4,675,187; and 4,837,206. These publications refer to anti-tumor agents and antibiotics which may be used as the pharmaceutical compound of the target.

The present invention provides antisense nucleic acids (including ribozymes), which may be used to inhibit expression of Cyr61, particularly to suppress Cyr61 effects on cell proliferation. An “antisense nucleic acid” is a single stranded nucleic acid molecule or oligonucleotide which, on hybridizing under cytoplasmic conditions with complementary bases in an RNA or DNA molecule, inhibits the latter's role. If the RNA is a messenger RNA transcript, the antisense nucleic acid is a countertranscript or mRNA-interfering complementary nucleic acid. As presently used, “antisense” broadly includes RNA-RNA interactions, RNA-DNA interactions, ribozymes and RNase-H mediated arrest. Antisense nucleic acid molecules can be encoded by a recombinant gene for expression in a cell (e.g., U.S. Pat. Nos. 5,814,500 and 5,811,234), or alternatively they can be prepared synthetically (e.g., U.S. Pat. No. 5,780,607). Also contemplated are vectors which include these oligonucleotides or antisense constructs.

### Compounds

### Steroids

A “sex steroid” refers to a class of hormonal substances that may be secreted from reproductive organs and glands. Sex steroids include, but are not limited to, estrogenic compounds, progestational compounds, and androgenic compounds.

Estrogenic compounds are described, for example, in the 11th edition of “Steroids” from Steraloids Inc., Wilton N. H. Non-steroidal estrogens described therein are included, as well. Other compounds included are derivatives, metabolites, and precursors. Also included are mixtures of more than one compound. Examples of such mixtures are provided in Table II of U.S. Pat. No. 5,554,601 (see column 6). Examples of estrogens either alone or in combination with other agents are provided, e.g., in U.S. Pat. No. 5,554,601.

β-estrogen is the β-isomer of estrogenic compounds. α-estrogen is the α-isomer of estrogen components. The term “estradiol” is either α- or β-estradiol unless specifically identified. The term “E2” is synonymous with 17β-estradiol.

Preferably, a non-feminizing estrogenic compound is used herein. Such a compound has the advantage of not causing uterine hypertrophy and other undesirable side effects, and thus, can be used at a higher effective dosage. Examples of non-feminizing estrogen include Raloxifene (Evista; Eli Lilly), Tamoxifen (Nolvadex; Astra Zeneca), and other selective estrogen receptor modulators.

Progestational compounds are described, for example, in the 9^{th} edition of “The Pharmacological Basis of Therapeutics” from McGraw-Hill, New York, N.Y. Progestin compounds, for example, include progestins containing the 21-carbon skeleton and the 19-carbon (19-nortestosterone) skeleton. Non-steroidal progestin compounds, derivatives, precursors, and metabolites are also contemplated herein.

Androgenic compounds are described, for example, in the 9^{th} edition of “The Pharmacological Basis of Therapeutics”. Androgens include, for example, testosterones containing the 17-carbon skeleton. Non-steroidal testosterone compounds, derivatives, precursors, and metabolites also are contemplated herein.

In addition, certain compounds, such as the androgen testosterone, can be converted to estradiol in vivo.

### Growth Factors

Growth factors are a class of proteins that are involved in stimulation of cell division. These proteins interact with cell surface receptors to induce transcription factors to promote cell survival. Growth factor receptors signal through the Ras pathway, a highly conserved signal transduction pathway. The Ras pathway functions to promote cell survival in radiation therapy, and genetic changes in this pathway which produce constitutively activate intracellular survival pathways are often associated with the development of cancer.

Growth factors include, for example, small molecule compounds that interact with growth factor receptors to produce the same effects as observed with growth factor peptides. Other compounds included are derivatives, metabolites, and precursors of endogenous growth factors. In specific embodiments of the present invention, specific growth factors that are used include, but are not limited to, epidermal growth factor, heparin binding epidermal growth factor, and basic fibroblastic growth factor.

### Assay System

Any cell assay system that allows for assessing functional activities of sex steroid, non-steroid, and growth factor receptor agonists and antagonists is contemplated by the present invention. In a specific embodiment, the assay can be used to identify compounds that interact with specific isoforms of sex steroid receptors to regulate Cyr61 transcription and translation, which can be evaluated by assessing the effects of a test compound, which modulates Cyr61 mRNA transcription and Cyr61 translation.

Any convenient method permits detection of the expressed product. For example, the invention provides Northern blot analysis for detecting Cyr61 mRNA product. The methods comprise, for example, the steps of fractionating total cellular RNA on an agarose gel, transferring RNA to a solid support membrane, and detecting a DNA-RNA complex with a labeled DNA probe, wherein the DNA probe is specific for a particular nucleic acid sequence of Cyr61 under conditions in which a stable complex can form between the DNA probe and RNA components in the sample. Such complexes may be detected by using any suitable means known in the art, wherein the detection of a complex indicates the presence of Cyr61 in the sample.

Typically, immunoassays use either a labeled antibody or a labeled antigenic component (e.g., that competes with the antigen in the sample for binding to the antibody). Suitable labels include without limitation enzyme-based, fluorescent, chemiluminescent, radioactive, or dye molecules. Assays that amplify the signals from the probe are also known, such as, for example, those that utilize biotin and avidin, and enzyme-labelled immunoassays, such as ELISA assays.

### In Vitro Screening Methods

Candidate agents are added to in vitro cell cultures of host cells, prepared by known methods in the art, and the level of Cyr61 mRNA and/or protein are measured. Various in vitro systems can be used to analyze the effects of a new compound on Cyr61 transcription and translation. Preferably, each experiment is performed more than once, such as, for example, in triplicate at multiple different dilutions of compound.

The host cell screening system of the invention permits two kinds of assays: direct activation assays (agonist screen) and inhibition assays (antagonist screen). An agonist screen involves detecting changes in the level of expression of the gene by the host cell contacted with a test compound; generally, gene expression increases. If the Cyr61 gene is expressed, the test compound has stimulated Cyr61 transcription via receptor interaction.

An antagonist screen involves detecting expression of the reporter gene by the host cell when contacted with an Cyr61 regulatory compound. If Cyr61 expression is decreased, the test compound is a candidate antagonist. If there is no change in expression of the reporter gene, the test compound is not an effective antagonist.

The assay system described here also may be used in a high-throughput primary screen for agonists and antagonists, or it may be used as a secondary functional screen for candidate compounds identified by a different primary screen, e.g., a binding assay screen that identifies compounds that interact with the receptor and affect Cyr61 transcription.

### In Vivo Testing Using Transgenic Animals

Transgenic animals, and preferably mammals, can be prepared for evaluating the molecular mechanisms of Cyr61. Preferably, for evaluating compounds for use in human therapy, the animals are “humanized” with respect to Cyr61. Such mammals provide excellent models for screening or testing drug candidates. The term “transgenic” usually refers to animal whose germ line and somatic cells contain the transgene of interest, i.e., Cyr61. However, transient transgenic animals can be created by the ex vivo or in vivo introduction of an expression vector of the invention. Both types of “transgenic” animals are contemplated for use in the present invention, e.g., to evaluate the effect of a test compound on Cyr61 or Cyr61 activity.

Thus, human Cyr61, “knock-in” mammals can be prepared for evaluating the molecular biology of this system in greater detail than is possible with human subjects. It is also possible to evaluate compounds or diseases on “knockout” animals, e.g., to identify a compound that can compensate for a defect in Cyr61 or Cyr61 activity. Both technologies permit manipulation of single units of genetic information in their natural position in a cell genome and to examine the results of that manipulation in the background of a terminally differentiated organism.

Although rats and mice, as well as rabbits, are most frequently employed as transgenic animals, particularly for laboratory studies of protein function and gene regulation in vivo, any animal can be employed in the practice of the invention.

A “knock-in” mammal is a mammal in which an endogenous gene is substituted with a heterologous gene (Roemer et al., New Biol. 3:331, 1991). Preferably, the heterologous gene or regulation system is “knocked-in” to a locus of interest, either the subject of evaluation(in which case the gene may be a reporter gene; see Elefanty et al., Proc Natl Acad Sci USA 95:11897,1998) of expression or function of a homologous gene, thereby linking the heterologous gene expression to transcription from the appropriate promoter. This can be achieved by homologous recombination, transposon (Westphal and Leder, Curr Biol 7:530, 1997), using mutant recombination sites (Araki et al., Nucleic Acids Res 25:868, 1997) or PCR (Zhang and Henderson, Biotechniques 25:784, 1998). See also, Coffman, Semin. Nephrol. 17:404, 1997; Esther et al, Lab. Invest. 74:953, 1996; Murakami et al., Blood Press. Suppl. 2:36, 1996.

A “knockout mammal” is an mammal (e.g., mouse) that contains within its genome a specific gene that has been inactivated by the method of gene targeting (see, e.g., U.S. Pat. Nos. 5,777,195 and 5,616,491). A knockout mammal includes both a heterozygote knockout (i.e., one defective allele and one wild-type allele) and a homozygous mutant. Preparation of a knockout mammal requires first introducing a nucleic acid construct that will be used to suppress expression of a particular gene into an undifferentiated cell type termed an embryonic stem cell. This cell is then injected into a mammalian embryo. A mammalian embryo with an integrated cell is then implanted into a foster mother for the duration of gestation. Zhou, et al. (Genes and Development, 9:2623-34, 1995) describes PPCA knock-out mice.

The term “knockout” refers to partial or complete suppression of the expression of at least a portion of a protein encoded by an endogenous DNA sequence in a cell. The term “knockout construct” refers to a nucleic acid sequence that is designed to decrease or suppress expression of a protein encoded by endogenous DNA sequences in a cell. The nucleic acid sequence used as the knockout construct is typically comprised of (1) DNA from some portion of the gene (exon sequence, intron sequence, and/or promoter sequence) to be suppressed and (2) a marker sequence used to detect the presence of the knockout construct in the cell. The knockout construct is inserted into a cell, and integrates with the genomic DNA of the cell in such a position so as to prevent or interrupt transcription of the native DNA sequence. Such insertion usually occurs by homologous recombination (i.e., regions of the knockout construct that are homologous to endogenous DNA sequences hybridize to each other when the knockout construct is inserted into the cell and recombine so that the knockout construct is incorporated into the corresponding position of the endogenous DNA). The knockout construct nucleic acid sequence may comprise (1) a full or partial sequence of one or more exons and/or introns of the gene to be suppressed, (2) a full or partial promoter sequence of the gene to be suppressed, or (3) combinations thereof. Typically, the knockout construct is inserted into an embryonic stem cell (ES cell) and is integrated into the ES cell genomic DNA, usually by the process of homologous recombination. This ES cell is then injected into, and integrates with, the developing embryo. However, the invention does not require any particular method for preparing a transgenic animal.

Generally, for homologous recombination, the DNA will be at least about 1 kilobase (kb) in length and preferably 3-4 kb in length, thereby providing sufficient complementary sequence for recombination when the construct is introduced. Transgenic constructs can be introduced into the genomic DNA of the ES cells, into the male pronucleus of a fertilized oocyte by microinjeciton, or by any methods known in the art, e.g., as described in U.S. Pat. Nos. 4,736,866 and 4,870,009, and by Hogan et al., Transgenic Animals: A Laboratory Manual, 1986, Cold Spring Harbor. A transgenic founder animal can be used to breed other transgenic animals; alternatively, a transgenic founder may be cloned to produce other transgenic animals.

Included within the scope of this invention is a mammal in which two or more genes have been knocked out or knocked in, or both. Such mammals can be generated by repeating the procedures set forth herein for generating each knockout construct, or by breeding to mammals, each with a single gene knocked out, to each other, and screening for those with the double knockout genotype.

Regulated knockout animals can be prepared using various systems, such as the tet-repressor system (see U.S. Pat. No. 5,654,168) or the Cre-Lox system (see U.S. Pat. Nos. 4,959,317 and 5,801,030).

### Cloning and Expression of Cvr61

The present invention contemplates analysis and isolation any antigenic fragments of Cyr61 from any source, preferably human. It further contemplates expression of functional or mutant Cyr61 protein for evaluation, diagnosis, or therapy.

Conventional molecular biology, microbiology, and recombinant DNA techniques within the skill of the art may be employed in the use of this invention. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

### Methods of Diagnosis-Upregulation

According to the present invention, upregulation of Cyr61 mRNA or protein can be detected to diagnose a Cyr61 associated disease, such as increased susceptibility to breast cancers. The various methods for detecting such upregulation of Cyr61 mRNA or protein expression are well known in the art and have been discussed earlier. Methods of detection include, but are not limited to, Northern blots, in situ hybridization studies, Western blots, ELISA, radioimmunoassay, “sandwich” immunoassays, immunoradiometric assays, gel diffusion precipitation reactions, immunodiffusion assays, in situ immunoassays (using colloidal gold, enzyme or radioisotope labels, for example),precipitation reactions, complement fixation assays, immunofluorescence assays, protein A assays, and immunoelectrophoresis assays, etc.

### Nucleic Acid Assays

The DNA may be obtained from any cell source. DNA is extracted from the cell source or body fluid using any of the numerous methods that are standard in the art. It will be understood that the particular method used to extract DNA will depend on the nature of the source. Generally, the minimum amount of DNA to be extracted for use in the present invention is about 25 pg (corresponding to about 5 cell equivalents of a genome size of 4×10⁹ base pairs). Sequencing methods are described in detail, supra.

In another alternate embodiment, RNA is isolated from biopsy tissue using standard methods well known to those of ordinary skill in the art such as guanidium thiocyanate-phenol-chloroform extraction (Chomocyznski et al., Anal. Biochem., 162:156, 1987). The isolated RNA is then subjected to coupled reverse transcription and amplification by polymerase chain reaction (RT-PCR), using specific oligonucleotide primers that are specific for a selected site. Conditions for primer annealing are chosen to ensure specific reverse transcription and amplification; thus, the appearance of an amplification product is diagnostic of the presence of a particular genetic variation. In another embodiment, RNA is reverse-transcribed and amplified, after which the amplified sequences are identified by, e.g., direct sequencing. In still another embodiment, cDNA obtained from the RNA can be cloned and sequenced to identify a mutation.

### Protein Assays

In an alternate embodiment, biopsy tissue is obtained from a subject. Antibodies that are capable of specifically binding to Cyr61 are then contacted with samples of the tissue to determine the presence or absence of a Cyr61 polypeptide specified by the antibody. The antibodies may be polyclonal or monoclonal, preferably monoclonal. Measurement of specific antibody binding to cells may be accomplished by any known method, e.g., quantitative flow cytometry, enzryme-linled or fluorescence-linked immunoassay, Western analysis, etc.

Immunoassay technology, e.g., as described in U.S. Pat. Nos. 5,747,274 and 5,744,358, and particularly solid phase “chromatographic” format immunoassays, are preferred for detecting proteins in blood or blood fractions.

### Pharmaceutical Compositions

The test compounds, salts thereof, antibodies, and antisense constructs may be formulated into pharmaceutical compositions. The pharmaceutical composition comprises a therapeutically, inhibiting preventing, or blocking effective amount of at least one of the above. This can be an amount effective to inhibit a sex steroid, a growth factor, or other factors that can increase Cyr61 expression or activity or the Cyr61 gene. The pharmaceutical compositions also typically include a pharmaceutically acceptable carrier (or dosing vehicle), such as ethanol, glycerol, water, and the like. Examples of such carriers and methods of formulation are described in Remington's Pharmaceutical Sciences, 18th edition (1990), Mack Publishing Company. The present invention also discloses pharmaceutical compositions that are composed on antibodies, neutralizing antibodies, conjugated antibodies, and antisense constructs. These pharmaceutical compositions comprise a therapeutically, inhibiting preventing, or blocking effective amount of at least one component. This can be an amount of the component effective to interact with the sex steroid or EGF receptor, the gene, or the protein. Conjugated antibodies can direct the secondary component to the targeted site.

The pharmaceutical composition may also include other additives, such as a flavorant, a sweetener, a preservative, a dye, a binder, a suspending agent, a colorant, a disintegrant, an excipient, a diluent, a lubricant, a plasticizer, or any combination of any of the foregoing. Suitable binders include, but are not limited to, starch; gelatin; natural sugars, such as glucose and beta-lactose; corn sweeteners; natural and synthetic guns, such as acacia, tragacanth, and sodium alginate; carboxymethylcellulose; polyethylene glycol; waxes; and the like. Suitable lubricants include, but are not limited to, sodium oleate, sodium stearate, magnesium stearate, sodium benzoate, sodium acetate, sodium chloride and the like. Suitable disintegrators include, but are not limited to, starch, methyl cellulose, agar, bentonite, xanthan gum and the like.

Suitable salts of the test compounds include, but are not limited to, acid addition salts, such as those made with acids, such as hydrochloric, hydrobromic, hydroiodic, perchloric, sulfuric, nitric, a phosphoric, acetic, propionic, glycolic, lactic pyruvic, malonic, succinic, maleic, fumaric, malic, tartaric, citric, benzoic, carbonic cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid; and salts made with saccharin. Other suitable salts of the compounds include, but are not limited to, alkali metal salts, such as sodium and potassium salts; alkaline earth metal salts, such as calcium and magnesium salts; and salts formed with organic ligands, such as quaternary ammonium salts.

Representative salts include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate salts of the compounds of the present invention.

The present invention includes prodrugs of the test compounds. Prodrugs include, but are not limited to, functional derivatives of the test compounds of the present invention which are readily convertible in vivo into the compounds of the present invention. Conventional procedures for the selection and preparation of suitable prodrug derivatives are described, for example, in “Design of Prodrugs”, ed. H. Bundgaard, Elsevier, 1985.

The pharmaceutical compositions may be formulated as unit dosage forms, such as tablets, pills, capsules, boluses, powders, granules, sterile parenteral solutions or suspensions, sterile I.V., sterile I.M., elixirs, tinctures, metered aerosol or liquid sprays, drops, ampoules, autoinjector devices or suppositories for oral, parenteral, intranasal, occular, mucosal, transdermal, bucal, topical, sublingual or rectal administration, or for administration by inhalation or insufflation, for example. The unit dosage form may be in a form suitable for sustained or delayed release, such as, for example, an insoluble salt of the compound, e.g. a decanoate salt, adapted to provide a depot preparation for intramuscular injection.

Solid unit dosage forms may be prepared by mixing the compound of the present invention with a pharmaceutically acceptable carrier and any other desired additives to form a solid preformulation composition. Examples of suitable additives for solid unit dosage forms include, but are not limited to, starches, such as corn starch; lactose; sucrose; sorbitol; talc; stearic acid; magnesium stearate; dicalcium phosphate; gums, such as vegetable gums; and pharmaceutical diluents, such as water. The solid preformulation composition is typically mixed until a homogeneous mixture of the compound of the present invention and the additives is formed, i.e., until the compound is dispersed evenly throughout the composition, so that the composition may be readily subdivided into equally effective unit dosage forms. The solid preformulation composition is then subdivided into unit dosage forms of the type described above.

Tablets or pills can also be coated or otherwise compounded to form a unit dosage form which has prolonged action, such as time release and sustained release unit dosage forms. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. The compound may be released immediately upon administration or may be formulated such that the compound is released in a sustained manner over a specified time course, such as, for example, 2-12 hours.

Liquid unit dosage forms include, but are not limited to, aqueous solutions, suitably flavoured syrups, aqueous or oil suspensions, and flavoured emulsions with edible oils, such as cottonseed oil, sesame oil, coconut oil or peanut oil, as well as elixirs and similar pharmaceutical vehicles. Suitable dispersing and suspending agents for aqueous suspensions include, but are not limited to, synthetic and natural gums, such as tragacanth, acacia, alginate, dextran, sodium carboxymethylcellulose, methylcellulose, polyvinyl-pyrrolidone and gelatin.

Suitable pharmaceutically acceptable carriers for topical preparations include, but are not limited to, alcohols, aloe vera gel, allantoin, glycerine, vitamin A and E oils, mineral oil, PPG2 myristyl propionate, and the like. Such topical preparations may be liquid drenches, alcoholic solutions, topical cleansers, cleansing creams, skin gels, skin lotions, and shampoos in cream or gel formulations (including, but not limited to aqueous solutions and suspensions). Typically, these topical preparations contain a suspending agent, such as bentonite, and optionally, an antifoaming agent. Generally, topical preparations contain from about 0.005 to about 10% by weight and preferably from about 0.01 to about 5% by weight of the compound, based upon 100% total weight of the topical preparation.

Pharmaceutical compositions of the present invention for administration parenterally, and in particular by injection, typically include an inert liquid carrier, such as water; vegetable oils, including, but not limited to, peanut oil, cotton seed oil, sesame oil, and the like; and organic solvents, such as solketal, glycerol formal and the like. A preferred liquid carrier is vegetable oil. These pharmaceutical compositions may be prepared by dissolving or suspending the compound of the present invention in the liquid carrier. Generally, the pharmaceutical composition for parenteral administration contains from about 0.005 to about 10% by weight of the compound of the present invention, based upon 100% weight of total pharmaceutical composition.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers include, but are not limited to, polyvinyl-pyrrolidone, pyran copolymer, polyhydroxypropylmethacryl-amidephenol, polyhydroxyethylaspartamidephenol, and polyethyl-eneoxideopolylysine substituted with palmitoyl residues. Furthermore, the compounds of the present invention may be coupled to biodegradable polymers for controlling the release of the compound, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

The pharmaceutical compositions of the present invention may be administered to an animal, preferably a human being, in need thereof to inhibit Cyr61 transcription or expression such as, for example, through activation of a steroid or growth factor receptor, or the like.

The effective amounts of the active agents of the pharmaceutical composition of the present invention may vary according to a variety of factors such as the individual's condition, weight, sex and age and the mode of administration. This amount of test compound can be determined experimentally by methods well-known in the art such as by establishing a matrix of dosages and frequencies and assigning a group of experimental subjects to each point in the matrix.

The compound of the present invention may be administered alone at appropriate dosages defined by routine testing in order to obtain optimal activity while minimizing any potential toxicity. In addition, co-administration or sequential administration of other active agents may be desirable.

The daily dosage of the compounds of the present invention may be varied over a wide range. For oral administration, the pharmaceutical compositions are preferably provided in the form of scored or unscored tablets for the symptomatic adjustment of the dosage to the patient to be treated. The dosage amount may be adjusted when combined with other active agents as described above to achieve desired effects. On the other hand, unit dosage forms of these various active agents may be independently optimized and combined to achieve a synergistic result wherein the pathology is reduced more than it would be if either active agent were used alone.

Advantageously, the pharmaceutical compositions may be administered in a single daily dose, or the total daily dosage may be administered in divided doses of two, three or four times daily.

For combination treatment with more than one active agent, where the active agents are in separate dosage formulations, the active agents can be administered concurrently, or they each can be administered at separately staggered times.

### EXAMPLES

The present invention will be better understood by reference to the following Examples, which are provided by way of exemplification and not by way of limitation.

### Example 1

### Estrogen and Progesterone Upregulate Cyr61 Transcription

T47D (progesterone responsive cells), MCF-7 (estrogen responsive cells), and MDA-MB-231 adenocarcinoma cell lines were obtained from ATCC (Rockville, Md.) and propogated in DMEM/F12/Ham's-10 media (GIBCO, Rockville, Md.) supplemented with 10% fetal bovine serum (FBS), 100 U/mL penicillin, 100 μg/mL streptomycin, and 2 mM Glutamax (GIBCO BRL; Rockville, Md.). For steroid treatments, adenocarcinoma cells were cultured in phenol-red free DMEM/F12 media supplemented with 2% charcoal stripped FBS (Clonetics, Inc; San Diego, Calif.).

T47D and MCF-7 cells were treated for varying time points (0-24 hours) with varying concentrations of test compounds. The pure ER antagonist ICI 182, 986 was co-incubated at a concentration of 1 μM with 10 nM 17β-estradiol in MCF-7 cells for 1.0 h prior to lysis in 10 nM guanidium isothiocynate detergent. The PR antagonist RU486 was co-incubated at a concentration of 1 μM with 1 μM R5020 in T47D cells for 4.0 h prior to lysis in 10 mM guanidium isothiocynate detergent.

Total cellular RNA was isolated from T47D and MCF-7 breast cancer cells by lysis in 250 μl of 10 mM guanidium isothiocynate detergent followed by 250 μl of phenol/chloroform extraction for 5 minutes at 25° C. Subsequently, total cellular RNA (20 μl) was subjected to electrophoresis in an 1% agarose gel containing IM formaldehyde in 10 mM MOPS buffer for 3 hours at 100 V at 25° C. Separated RNA transcripts were transferred onto nylon membranes by capillary electrophoresis in 10×SSC at 25° C. for 18 hours, and subsequently prehybridized at 60° C. in RapidHyb hybridization solution (Amersham, Arlington Heights, Ill.). A 0.41 kb human Cyr61 a cDNA fragment was radiolabeled with [α-³²P]-dCTP (3,000 Ci/mmol) using the rando-primer technique (Rediprime II, Amersham) and used as the hybridization probe. The radiolabeled probe (1×10⁶ cpm/ml) was hybridized to membranes for 4 hours at 60° C. Membranes were washed twice in 1×SSPE (0.15 M NaCl, 1 μM EDTA, and 0.01 M sodium phosphate, pH 7.4) and 0.1% SDS for 15 minutes at 25° C., followed by a final wash in 0.1×SSPE and 0.1% SDS for 5 minutes at 60° C. Densiometric analysis of Cyr61 mRNA levels was accomplished with Molecular Dynamics phosphorimager and image quantification software (Amersham Pharmacia Biotech, Piscataway, N.J.). Relative levels of Cyr61 were normalized to glyceraldehyde-3-phosphate dehydrogenase (GAPDH) after reprobing membranes with a ³²P-radiolabeled oligonucleotide according to manufacturers protocol (endlabeling lcit, GibcoBRL, Rockville, Md.).

Statistical analysis was performed using SAS statistical software (SAS Inc., Cary, N.C.) for significance using a one-way analysis of variance (ANOVA). Multicomparison significance level for the ANOVA was a p-value equal to or less than 0.05. If significance was achieved, a Scheffe's F test was performed.

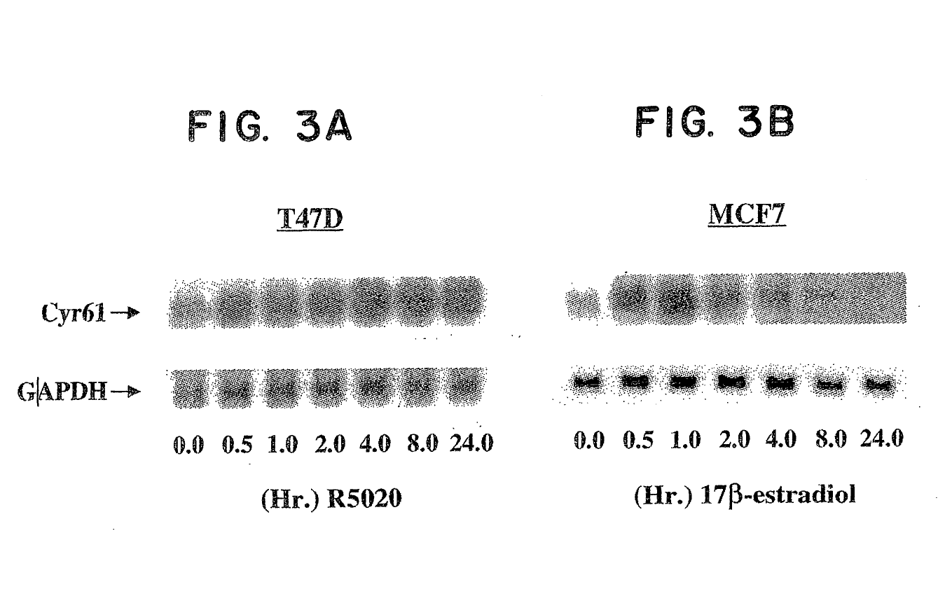

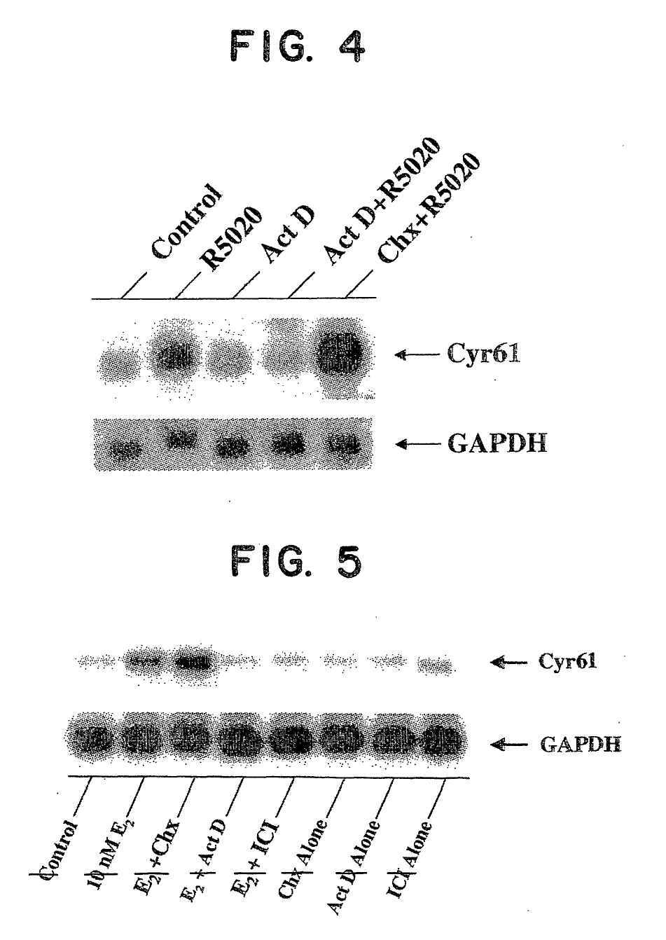

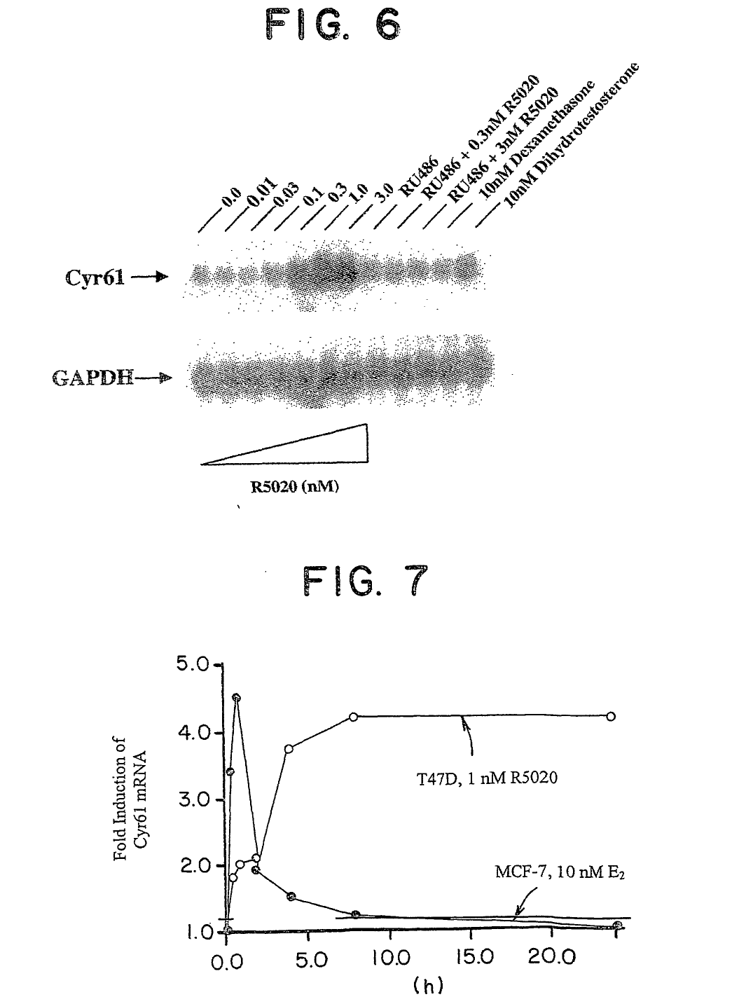

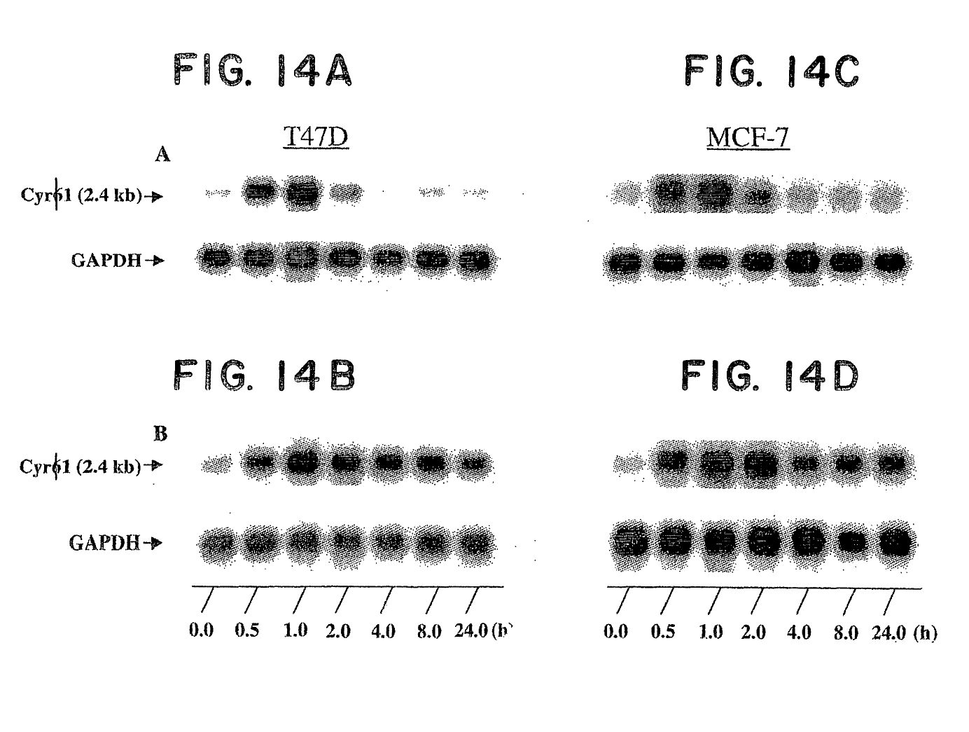

Results are shown in FIGS. 3-7 and 14. FIG. 3 shows that estrogen and progesterone receptor ligands induce up regulation of Cyr61 mRNA in T47D and MCF-7 cells, respectively. FIG. 4 shows that upregulation of Cyr61 by R5020 in T47D cells occurs at the transcriptional level since R5020 effects were fully blocked by the transcription inhibitor actinomycin D, but not the protein synthesis inhibitor cyclohexamide. FIG. 5 shows that upregulation of Cyr61 by 17β-estradiol in MCF-7 cells occurs at the transcriptional level since 17β-estradiol effects were fully blocked by the transcription inhibitor actinomycin D, but not the protein synthesis inhibitor cyclohexamide. FIG. 6 shows that R5020 induction of Cyr61 mRNA transcription occurred in a dose-dependent manner and was progesterone receptor specific, since the progesterone receptor antagonist, RU486, fully blocked the observed effects and other steroids had little to no effect. FIG. 7 shows the time course of mRNA induction in cells treated with estrogen and progesterone receptor ligands. Stimulation of Cyr61 transcription by estrogen ligands occurred earlier than Cyr61 transcription by progesterone ligands. FIG. 14 shows that R5020 and 17β-estradiol stimulates the effects of EGF on mRNA transcription in T47D and MCF-7.

### Example 2

### Estrogen and Progesterone Upregulate Cyr61 Protein Expression

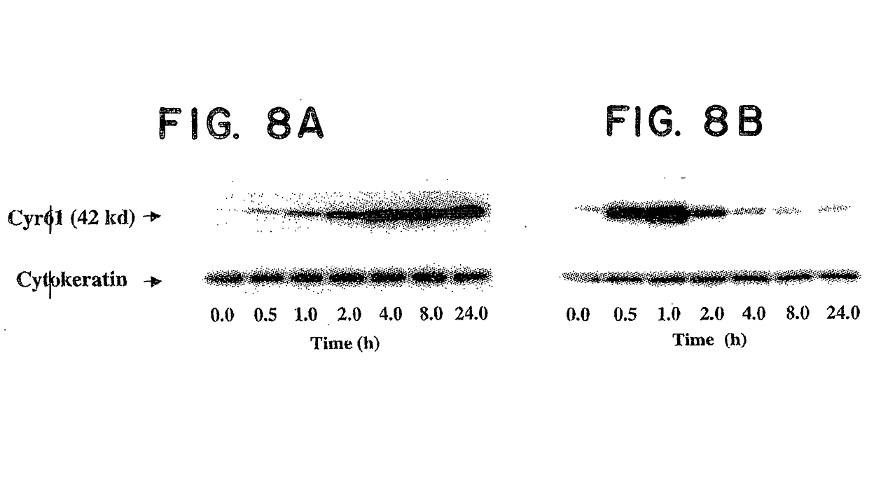

T47D and MCF-7 adenocarcinoma cell lines were maintained and propagated as described in Example 1. Cells were treated for varying time points (0-24 hours) with varying concentrations of test compounds. Cells were homogenized in 50 mM Tris-HCl (pH=8.0) with 250 mM NaCl, 1.0% Nonidet P-40, 1.0% Triton-X 100, 2.0% sodium dodecyl sulfate, 0.5% deoxycholate, 1 mM ethylenediaminetetraacetic acid, and a protease inhibitor cocktail containing 10 μg/mL pepstatin, aprotinin, and leupeptin each (Sigma; St. Louis, Mo.). Protein extracts (20 μg) were subjected to SDS-polyacrylamide gel electrophoresis under reducing conditions in 10% bis-acrylamide gels at 100 V for 3 hrs at 25° C. Proteins were electrophoretically transferred to polyvinyl difluoride membrane (Immobilon-P, Biorad, Redding, Calif., USA) in 500 mls of 100 mM Tris-Glycine/1-% MeOH buffer at 50 mA for 18 hrs. Membranes were blocked with 5% dry milk on TBS/0.1% Tween-20 (TBST), and incubated with anti-Cyr61 pAb (10 μg/ml) for 1 hr. at room temperature. Following primary antibody incubation, membranes were washed 4×10 mins. with 50 mls of 1×TBST and subsequently incubated with 10 μgs/ml donkey anti-rabbit IgG antibody conjugated to horseradish peroxidase (HRP) for 1 hr. at room temperature. Secondary antibody detection was determined by an enhanced chemilurninescence detection kit (Amersham Biotech, Piscataway, N.J.) at room temperature for 5 min. In order to normalize protein levels, Cyr61 western blots were subsequently reprobed with 1 μg/ml anti-cytokeratin monoclonal antibody (Sigma, Inc.; St. Louis, Mo) and 1 μg/ml of donkey anti-mouse secondary antibody conjugated to HRP (Amersham Biotech, Piscataway, N.J.,) for 1 hr. at room temperature. Cyr61 protein levels were quantified by densiometric analysis using a Biorad molecular imager (Biorad Laboratories, Hercules, Calif.). Numerical values were based on the relative optical density (OD) of the band. Protein levels were normalized to the total level of cytokeratin in each sample. Statistical analysis was performed as described above. Results are shown in FIG. 8. FIG. 8 shows increased Cyr61 protein expression is observed after stimulation of cells with estrogen and progestin ligands.

### Example 3

### Cyr61 is Upregulated in Human Breast Cancer Tumors Classified as ER+, PR+ and/or EGFR+

Breast tumor biopsies and matched normal mammary tissues were obtained from Clinomics (Pittsfield, Mass.) following informed patient consent and internal review borad approval. Patients (n=40) were between the ages of 42-68 and diagnosed with stage 1I invasive ductal carcinoma following histological examination. All breast tumor classifications were performed by Clinomics, Inc. (Pittsfield, Mass.) utilizing standard immunohistochemical techniques. Tumors were classified as estrogen receptor (ER), progesterone receptor (PR), and epidermal growth factor receptor (EGFR) positive (n-20) or ER and PR negative and EGFR positive (n=20). Briefly, formalin-fixed tissue sections were deparaffinized in 100% Xylene for 10 min at room temperature and rehydrated through a 100%-30% EtOH gradient at room temperature. Tissue sections were subsequently incubated with either anti-ER (Santa Cruz Technologies, Santa Cruz, Calif.), anti-PR (Santa Cruz Technologies, Santa Cruz, Calif.) or anti-EGFR (Sigma Immunochernicals, St. Louis, Mo.) monoclonal antibodies for 1 hr. at room temperature. Tissue sections were washed 2×10 min at room temperature in Tris-Buffered Saline (TBS) and incubated with goat anti-mouse secondary antibodies conjugated to horseradish peroxidase (HRP) for 1 hr at room temperature. Sections were washed again in TBS 2×10 min at room temperature and incubated with a chromagenic substrate (Vector Laboratories, Burlingame, Calif.) for colormetric detections. Tissue sections were counterstained with hematoxylin (Sigma Inc., St. Louis, Mo.), dehydrated in graded ethanol series, and mounted for viewing. Receptor positive tissues were identified by brown percipatates that were associated with the cell nucleus (ER and PR) or cell membranes (EGFR). Cyr61 protein extraction and data analysis was performed as described in Example 2.

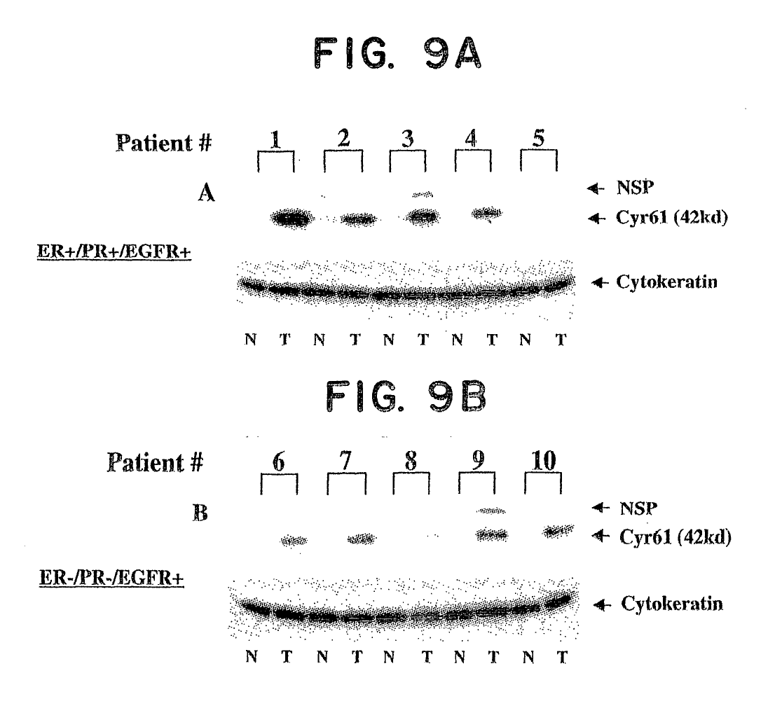

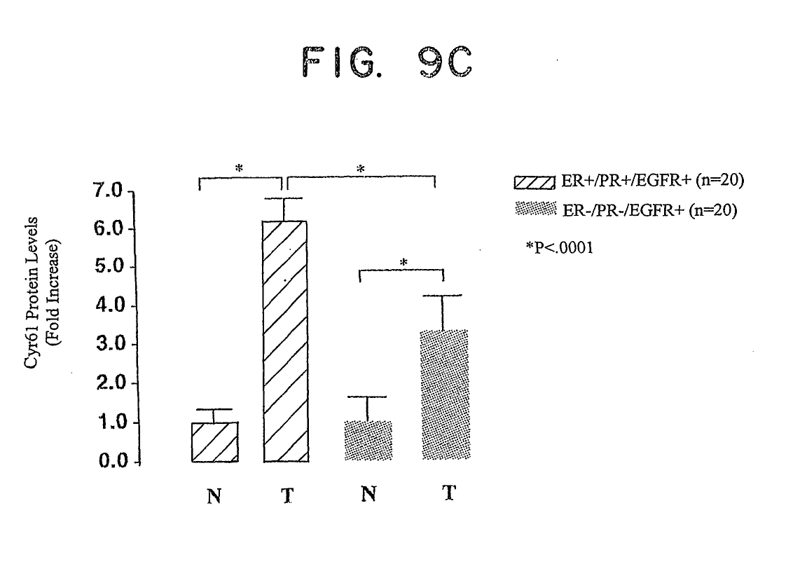

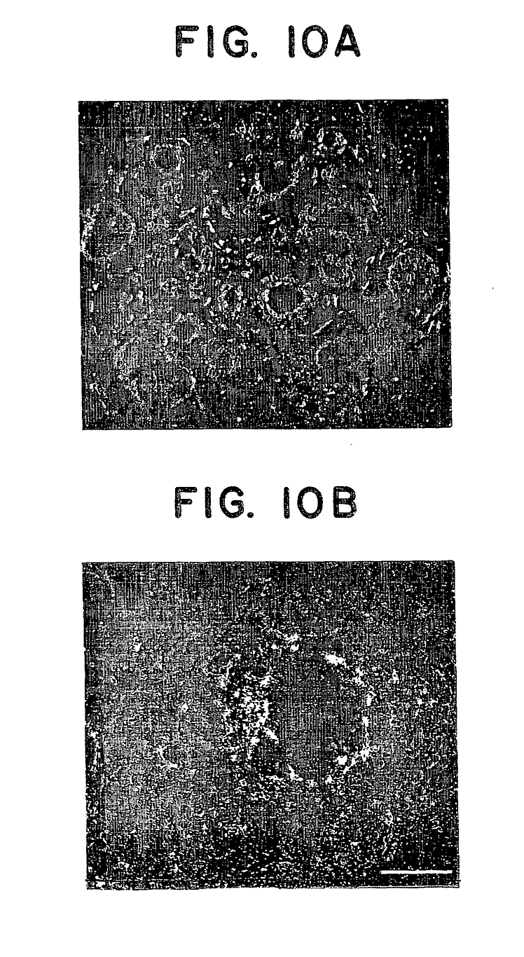

Results are shown in FIG. 9. FIG. 9 shows that tumors that are classified as ER+/PR+/EGFR+ display a higher level of Cyr61 protein when compared to tumors that are classified as ER−/PR−/EGFR+. Both ER+/PR+/EGFR+ and ER−/PR−/EGFR+ tumors displayed higher levels of Cyr61 protein than normal mammary cells.

### Example 4

### Differential Expression of Cyr61 in Breast Tumor Patients

Breast tumor biopsies were fixed in 10% neutral-buffered formalin. 0.28 kb human Cyr61 cDNA fragment was positionally cloned into the EcoRI and HindIII sites of pGEM4Zf-plasmid (Promega Corp.; Madison, Wis.) to generate pGEM4Zf-Cyr61. Radiolabeled ³⁵S-UTP sense and antisense cRNA transcripts were transcribed in vitro with T3 and T7 RNA polymerases, respectively, using Gemini Riboprobe system (Promega).

In situ hybridization studies were performed utilizing processed slides that were hybridized overnight with 100-150 μl of antisense or sense riboprobes at 4.7×10⁶ DPM/slide in 50% formamide hybridization mixture including 5% dextran sulfate and 100 dithiothreitol (DTT) at 55° C. in a humidified chamber containing 50% formamide/600 mM NaCl. Slides were washed 3 times in 2×SSC (0.3 M NaCl, 0.03 M sodium citrate, pH=7.0)/10 mM DTT at room temperature. The washes were followed by RNase A (20 μg/ml) treatment for 30 minutes at 37° C. and then washed for 15 minutes in 0.1×SSC at room temperature. Slides were further washed with 0.1×SSC to remove nonspecific label and dehydrated with a graded series of alcohol:ammonium acetate (70%, 95%, and 100%). Air-dried slides were exposed to X-ray film (Amerasham Inc., Piscataway, N.J.) for 3 days for preliminary examination and then dipped in NTB2 nuclear emulsion (Eastman Kodak; Rochester, N.Y.), which was diluted 1:1 with 600 mM ammonium acetate. Slides were exposed for 31 days in light-tight, black dessicated boxes, photographically processed, and then stained in cresyl violet and coverslipped.

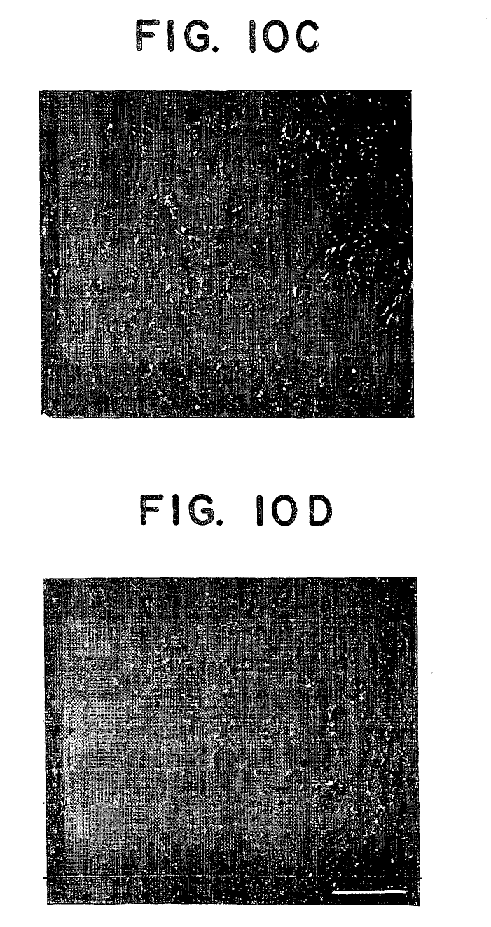

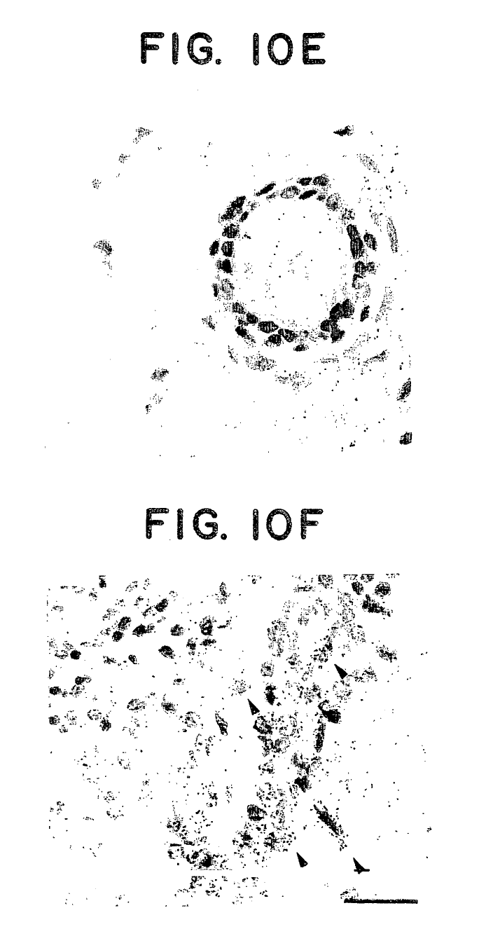

Results are shown in FIG. 10. FIG. 10 shows in situ hybridization studies that show that Cyr61 levels are very low in normal breast cell, but are abundant in invading luminal epithelial cells within tumors.

### Example 5

### Cyr61 Neutralizing Antibodies Block Estrogen Steroid Mediated Cell-Proliferation

Two anti-Cyr61 polyclonal antisera were generated at the Louisiana State University Medical Center Core Facilities (Baton Rouge, La.) to peptides corresponding to amino acids 163-229 and amino acids 371-381 of the human Cyr61 protein. A cysteine was added to the N-terminus for coupling to carrier proteins. Peptides were synthesized using an automated phase peptide synthesizer using 9-fluorenylnethyloxycarbonyl (Fmoc) chemistry (PE biosystems 9050+). A Waters Delta Prep 400 preparative chromatography system, with a C18 Phenomenex Jupiter column (250×21.20 mm, 10μ diameter) equipped with a photo diode array detector was used to purify the peptide. A flow rate, through the column, in excess of 100 mL/min purified about 400-500 mgs of peptide. The identity and purity of the antigenic peptides was evaluated using a PE Biosystem DE-MALDI mass spectrometer. Peptides were subsequently coupled to heyhole-limpet hemocyanin and mixed with an equal volume of Complete and Incomplete Feund's Adjuvant.

The mixture was then injected into female New Zealand white rabbits (200 μg antigen and adjuvant mixture/rabbit). On days 14 and 28, rabbits were administered a booster injection that was the same size as the initial injection. On day 38, blood from rabbits was tested using an ELISA (using a Strepavidin/Biotin system) for antibody presence. If an increased antibody titer is required, rabbits were administered a booster injection that was the same sample size as the initial injection on day 42. Serum was collected from the rabbits on day 52 and frozen. Polyclonal antibodies were affinity purified by attaching the antigen to a stationary phase (Sulfo-Link Resin, Pierce) using the side chain of cysteine. Approximately 30 mL of serum was loaded through the column and then washed out to remove non-binding proteins. Antibodies were eluted with 3.5 M MgCl₂/ethyl glycol. Eluted proteins are dialyzed and then concentrated to approximately 1 mg/mL. Concentration is determined by OD at 280 mm.

T47D, MCF-7, and MDA-MB-231 adenocarcinoma cell lines were treated with test compounds in the presence or absence of 10 μg/mL anti-Cyr61 antibodies for 18 hours. For DNA synthesis studies, bromodeoxyuridine (BrdU) incorporation was measured using the BioTrak ELISA kit (Amersham; Arlington Heights, Ill.) according to manufacturer's directions. For cell proliferation studies, MCF-7 cells were cultured in phenol-red free DMEM/F12 media supplemented with 2% charcoal stripped FBS (Clonetics, Inc; San Diego, Calif.) and test compounds for 5 days in the presence or absence of 10 μg/mL of anti-Cyr61 antibodies at 37° C. in 5% CO₂. Following treatment, cells were incubated in of trypsin and disloged from the cell culture flask. These cells were combined with cells in the supernatant of the tissue culture media. The mixture was then counted in a Coulter Multisizer II counter (Coulter Corporation; Miami, Fla.).Experiments were performed in quadruplicates and numerical vaules were analyzed for statistical significance by a one-way analysis of variance (ANOVA) utilizing SAS statistical software (SAS Inc., Cary, N.C.). Mutlicomparison significance level for the ANOVA was a p-value equal to or less than 0.05. If significance was achieved, a Scheffe's F test was performed.

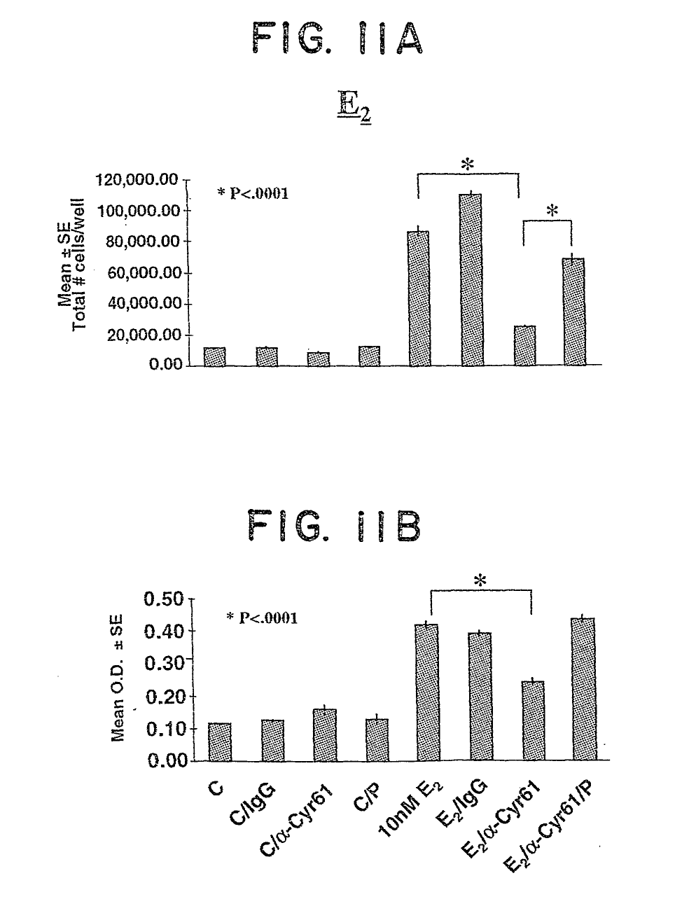

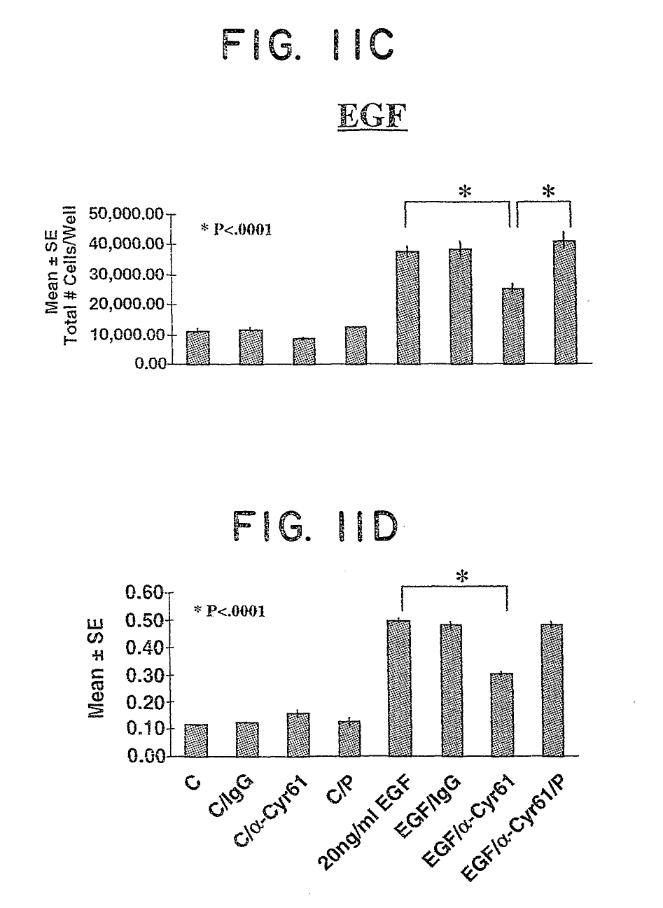

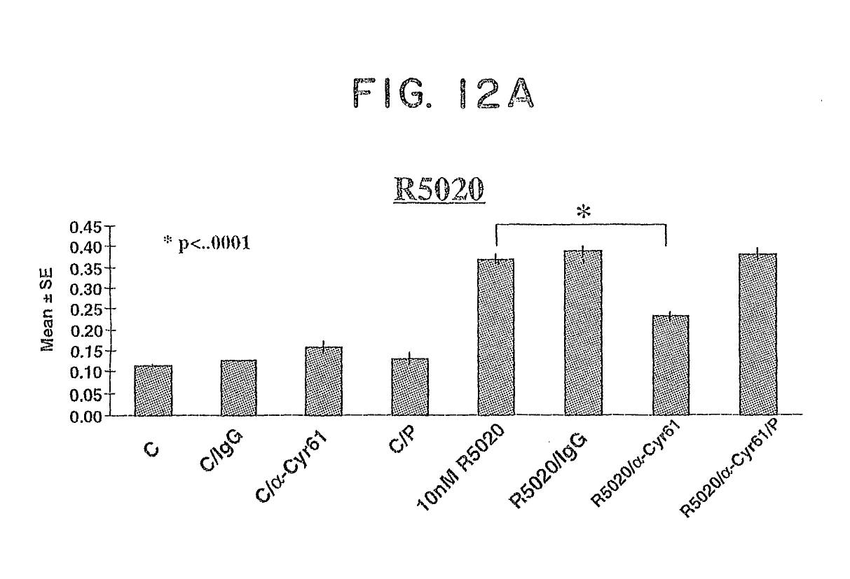

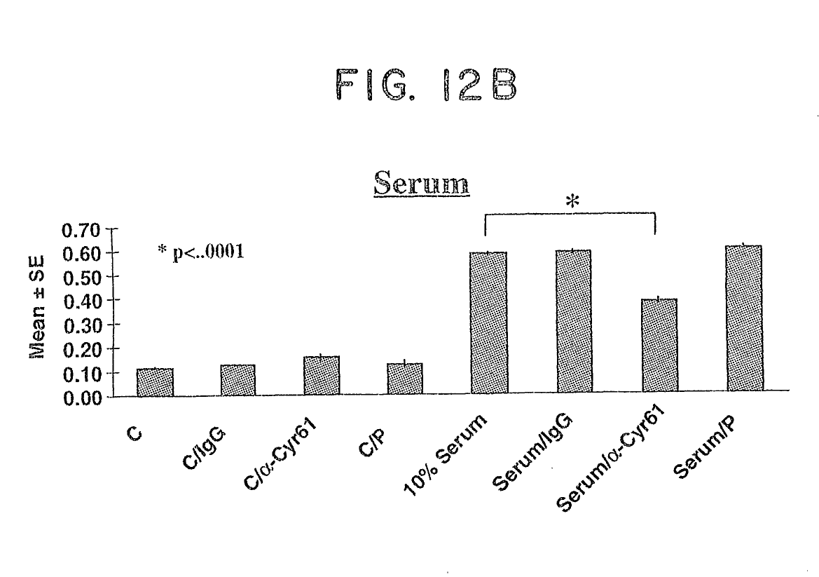

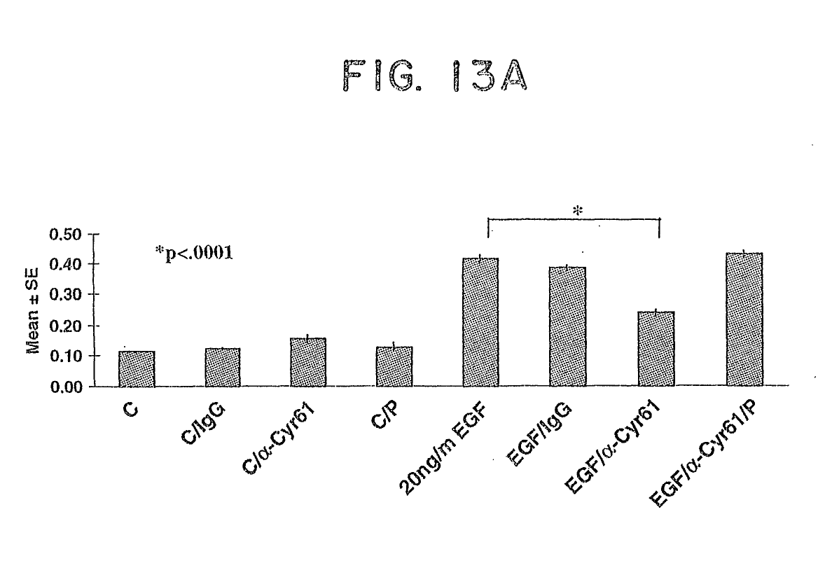

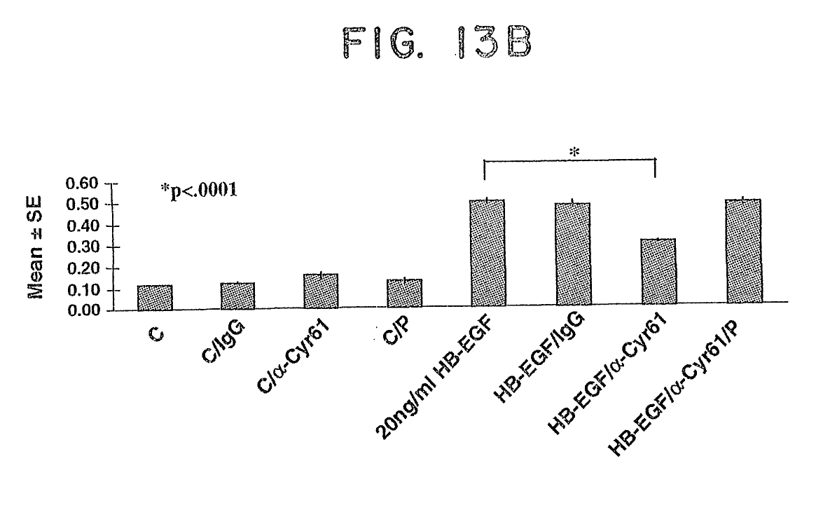

Results are shown in FIGS. 11-13. FIG. 11 shows that Cyr61 neutralizing antibody clocks estrogen and EGF-mediated stimulation of cell-proliferation and DNA-synthesis in MCF-7 cells. FIG. 12 shows that Cyr61 neutralizing antibody clocks progesterone and serum mediated stimulation of DNA-synthesis in T47D cells. FIG. 13 shows that Cyr61 neutralizing antibodies partially block EGF and HB-EGF-mediated stimulation of DNA synthesis in MDA breast cancer cells.

### Example 6

Androgens Upregulate Cyr61 Transcription

MDA-MB-453 and ZR-75-1 adenocarcinoma cell lines were obtained from ATCC (Rockville, Md.) and propagated in Dulbecco's Modified Eagles Medium low glucose (DMEM-LG) media containing 10% fetal bovine serum (FBS), 100 U/ml penicillin, 100 μg/ml streptomycin, and 2 mM Glutamax (Gibco BRL, Rockville, Md.). MDA-MB-231 adenocarcinoma cell lines were obtained from ATCC and propagated in DMEM/F12 Ham's-10 media containing 10% fetal bovine serum (FBS), 100 U/ml penicillin, 100 μg/ml streptomycin, and 2 mM Glutamax. For steroid and/or growth factor treatments, MDA-MB-453 and ZR-75-1 cells were cultured in phenol-red free DMEM-LG media and MDA-MB-231 cells were grown in DMENVF12 HAM's-10 media containing 2% charcoal stripped FBS (Hyclone Inc., Logan, Utah). Incubation and analysis were performed according to techniques described in Example 1.

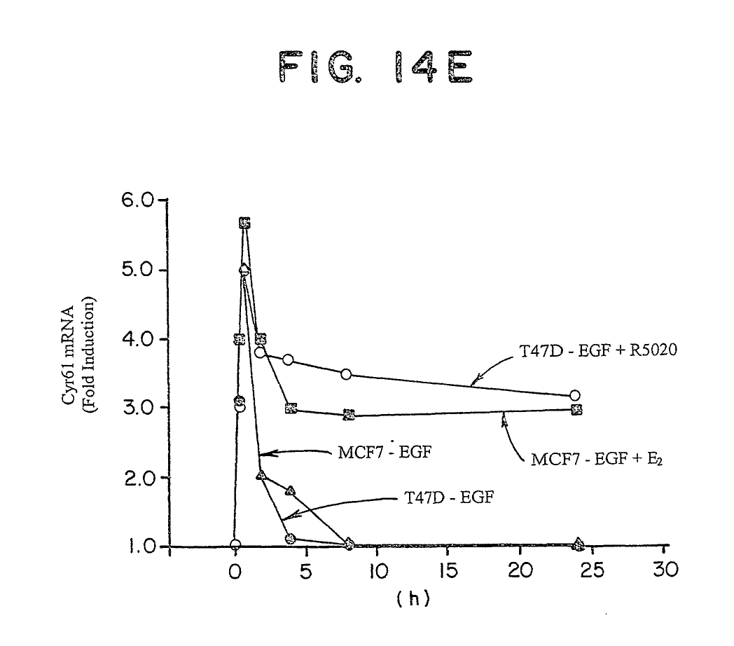

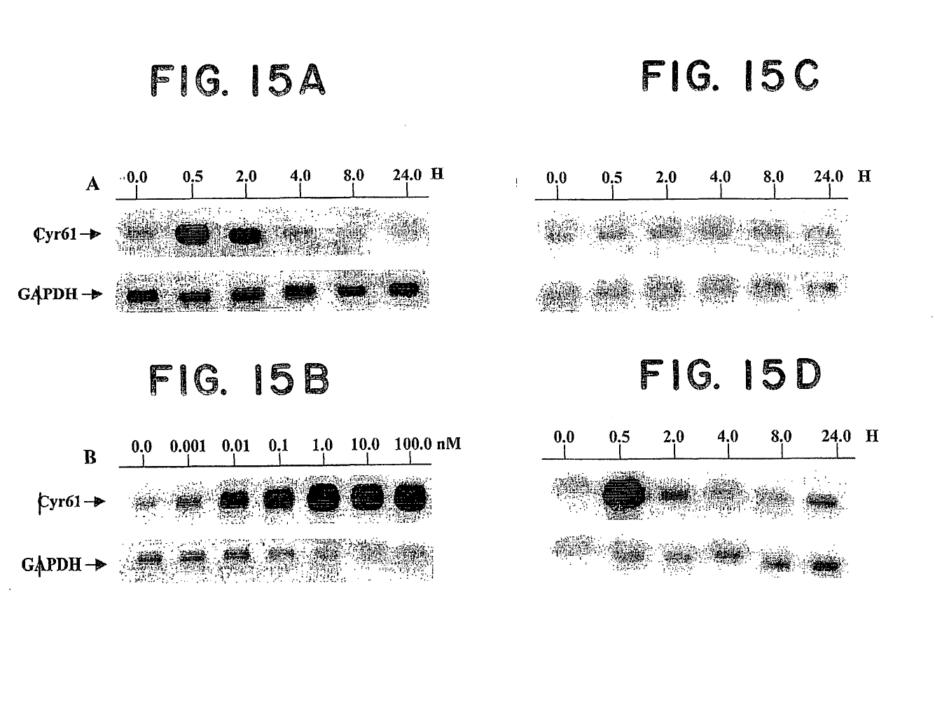

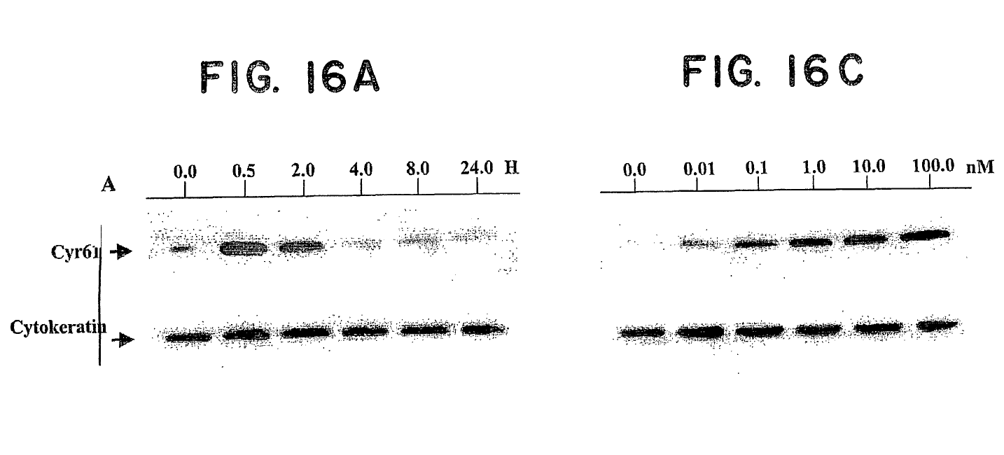

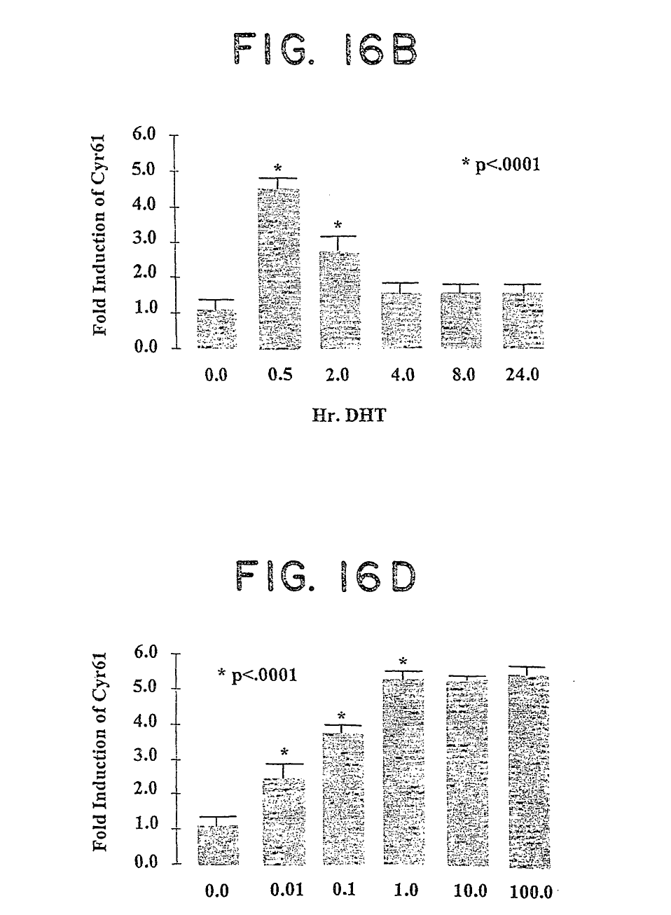

Results are shown in FIGS. 15-16. DHT induced Cyr61 mRNA and protein in an immediate early fashion in MDA-MB-453 but not in ZR-75-1 cells with maximum expression (8-fold) occurring within 0.5 h (FIGS. 15 and 16). The lack of induction of Cyr61 by DHT in ZR-75-1 cells was not due to aberrant genetic deletions or Cyr61 gene loss as EGF rapidly upregulated Cyr61 mRNA to maximal levels within 0.5 h (FIG. 15D). The upregulation of Cyr61 mRNA and protein by DHT in MDA-MB-453 cells was dose-dependent with maximal expression occurring at 1.0 nM (EC₅₀=˜0.3 nM) (FIGS. 15 and 16). Thus similar to growth factors and E2, Cyr61 is rapidly upregulated in an immediate early-fashion in MDA-MB-453 breast cancer cell line that overexpresses AR.

### Example 7

### Androgens Upregulate Cyr61 Transcription

Total cellular RNA was isolated from cultured adenocarcinoma cells by guanidium isothiocynate lysis followed by phenol/chloroform extraction. Subsequently, total cellular RNA (20 μg) was subjected to electrophoresis in an 1% agarose gel containing 1 M formaldehyde, and transferred onto nylon membranes by capillary electrophoresis. A 0.41 kb human Cyr61 cDNA fragment was radiolabeled with [α-³²P]-dCTP (3,000 Ci/mmol) using the random-primer technique (Rediprime II, Amersham Inc.) and used as the hybridization probe. Relative levels of Cyr61 were normalized to glyceraldehyde-3-phosphate dehydrogenase (GAPDH) after reprobing membranes with a ³²P-radiolabeled oligonucleotide according to manufacturer's protocol (endlabeling kit, GibcoBRL, Rockville, Md.).

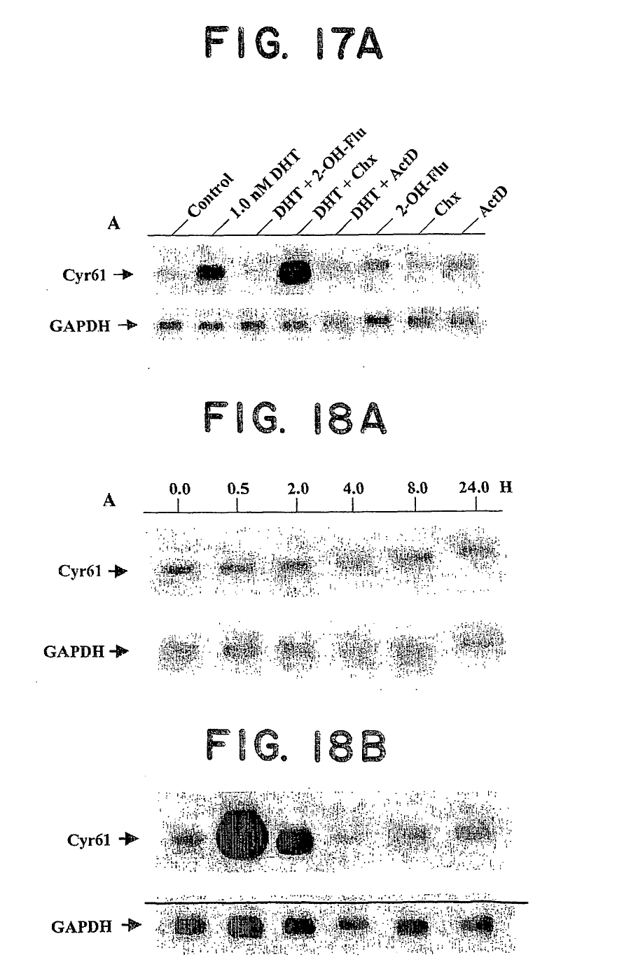

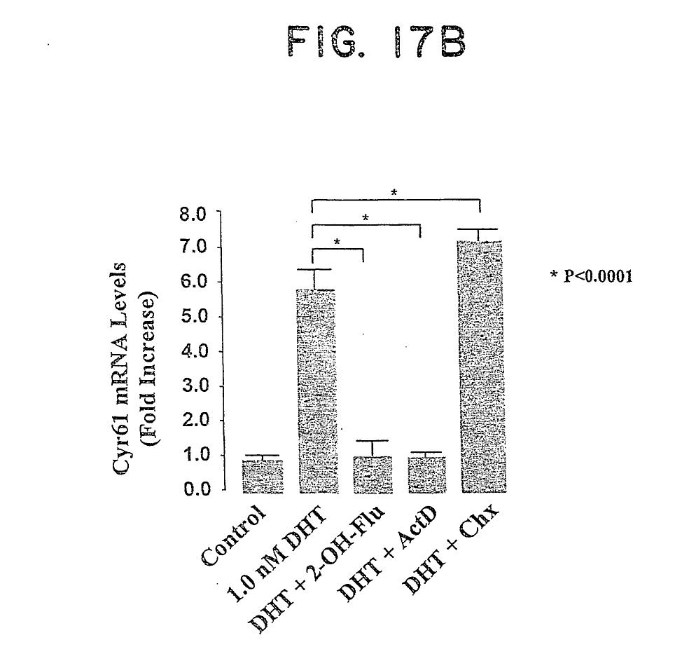

Results are shown in FIGS. 17-18. DHT induction of Cyr61 in MDAMB453 cells is specifically mediated through AR since the AR antagonist, 2-OH flutamide (2-OH-flu) completely abrogated expression (FIG. 17A). Additionally, inhibition of Cyr61 upregulation was not observed in MDA-MB-453 cells co-treated with 10 μg/ml cycloheximide (Chx) and 1 nM DHT, suggesting that de novo protein synthesis was not required for androgen induction (FIG. 17A). In contrast, significant overexpression of Cyr61 was observed upon co-treatment with Chx and DHT when compared to DHT treatment alone (FIG. 17A). These increases in mRNA levels are often observed with Chx coWO treatment, which may be due to the lack of synthesis of mRNA decay or turnover factors. However, co-treatment with 1 nM DHT and 1 μg/ml actinomycin D completely blocked the androgen induction, implying that AR mediates Cyr61 expression at the transcriptional level (FIGS. 17A and B). Furthermore, in AR-MDA-MB-231 adenocarcinoma cells expression of Cyr61 was not enhanced by DHT, but was rapidly induced upon EGF stimulation, demonstrating that AR was required for Cyr61 induction (FIGS. 18A and B) and that androgens did not function in a non-genomic capacity. Therefore, Cyr61 is primarily induced by androgens through AR in an immediate-early fashion in MDA-MB-453 cells at the transcriptional level.

### Example 8

### Cyr61 s Upregulated in Human Cancer Tumors Classified as AR+

Metastatic breast tumor biopsies and matched normal mammary tissue specimens were obtained from Clinomics Inc., (Pittsfield, Mass.) following informed patient consent and internal review board approval. Patients (n=20) were between the ages of 31-92 (average=65) and initially diagnosed with invasive ductal carcinoma prior to developing metastatic focal lesions following histological examination. In addition, tumors were classified as AR+ (n=10) or AR− (n=10) by immunohistochemical staining of formalin-fixed tumor biopsies using mono-specific anti-AR antibodies. Tissue specimens were immediately frozen in liquid nitrogen following surgery for protein extraction.

Tissue protein extracts were prepared from breast tumors and matched normal mammary tissue specimens by homogenization in 50 mM Tris, pH 8.0, 250 mM NaCl, 1.0% Nonidet P-40, 1.0% Trtion-X 100, 2.0% SDS, 0.5% deoxycholate, 1 mM EDTA, and protease inhibitor cocktail containing 10 μg/ml pepstatin, aprotinin, and leupeptin (Sigma-Aldrich). Protein extracts (20 μg) were subjected to SDS-polyacrylamide gel electrophoresis under reducing conditions in 10% bis-acrylamide and electrophoretically transferred to polyvinyl difluoride membrane (hmobilon-P, Biorad, Redding, Calif., USA). Membranes were blocked with 5% dry milk on TBS/0.1% Tween-20 (TBST), and incubated with anti-Cyr61 pAb (10 μg/ml). Primary antibody binding was detected using a Donkey antirabbit IgG antibody conjugated to horseradish peroxidase (HRP) and an enhanced chemiluminescence detection system (Amersham). All immunoblots were subsequently reprobed with 1 μg/ml of anti-pan-cytokeratin monoclonal antibodies (Sigma-Aldrich) to verify equivalent protein loading. All Western blotting was performed at least two times per treated lysate or breast tumor extract.

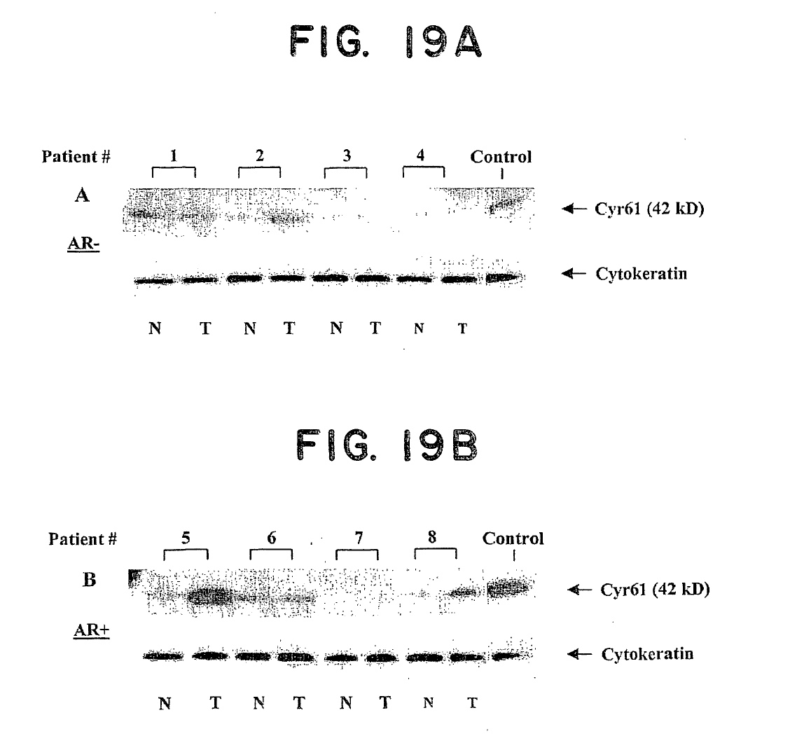

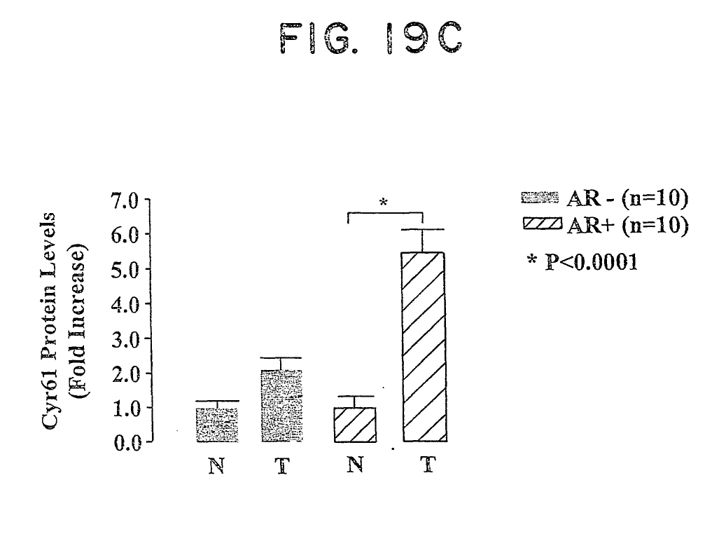

Results are shown in FIG. 19. Analysis of Cyr61 protein levels in AR-metastatic tumors detected overexpression in 1 out of 10 tissue extracts when compared to matched controls (FIGS. 19A and C). Interestingly, a 3.5-6 fold increase in Cyr61 protein levels was observed in 40% (4/10) of AR+ metastatic breast tumor tissue extracts compared to autologous controls (FIGS. 19B and C). Although further analysis with a larger patient population is necessary, it appears initially that upregulation of Cyr61 protein may correlate with AR positivity in vivo. The latter suggests that Cyr61 may be regulated by androgens or under the control of AR during tumor progression in vivo.

### Example 9

### Cyr61 Antibodies Block Androgen Mediated DNA-Synthesis and Cell-Proliferation

Polyclonal antibodies were raised to a 65 a.a. peptide corresponding to the central domain of Cyr61 which was selected based on a lack of homology to other CCN family members and published effectiveness of the antibodies in neutralizing bFGF-mediated DNA synthesis in human microvascular endothelial cells. The antibodies were affinity purified and assayed for specificity by western blot analysis using E₂-treated MCF-7 whole cell lysates. A major band was detected at 42 kD, which is the molecular weight of Cyr61, along with a minor band at 77 kD. For DNA synthesis, MDA-MB-453 cells were treated with either 1.0 nM DHT or 20 ng/ml EGF in the presence or absence of 10 μg/ml anti-Cvr61 antibodies for 18 h and bromodeoxyuridine (BrdU) incorporation was measured using the BioTrak ELISA kit (Amersham, Arlington Heights, Ill.) with a horseradish peroxidase reporter enzyme according to manufacturer's instructions.

For cell proliferation assays, MDA-MB-453 cells were cultured in 2% charcoal stripped FBS media containing 1.0 nM DHT or 20 ng/ml EGF for 10 days in the presence or absence of 10 μg/ml of anti-Cyr61 neutralizing antibodies at 37° C. in 5% CO₂, with treatment changes every other day. Following steroid or growth factor treatment, monolayers were trypsinized, combined with cells in the culture supernatant, and counted in a Coulter Multisizer II counter (Coulter Corporation, Miami, Fla.). All treatments were performed in quadruplicates and each experiment was repeated at least three times.

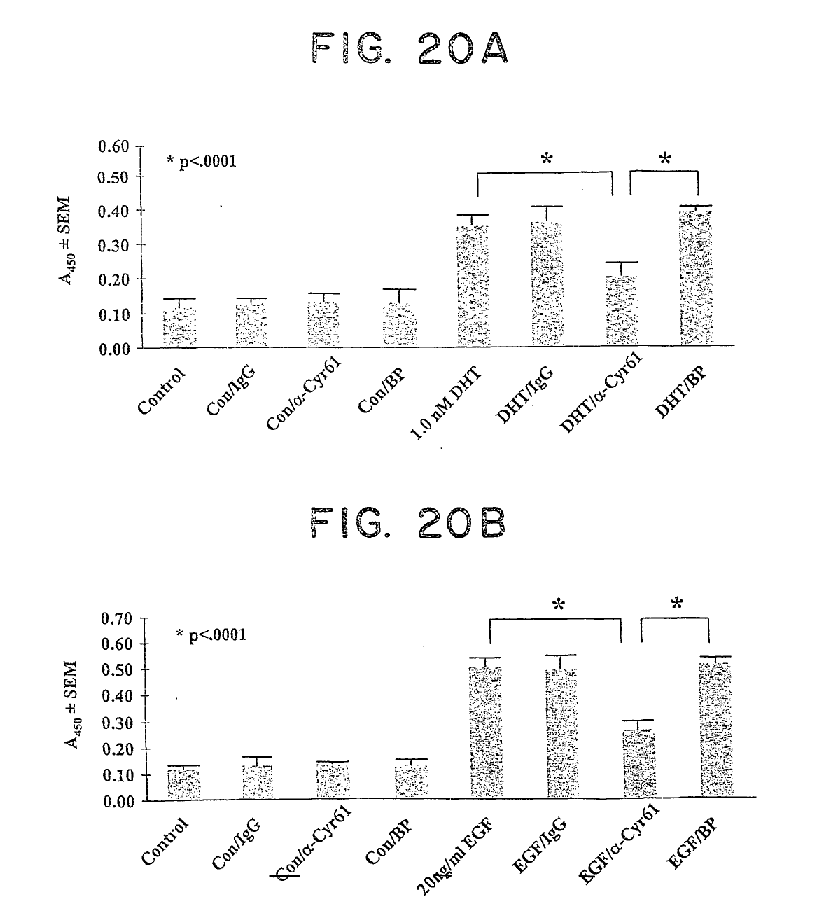

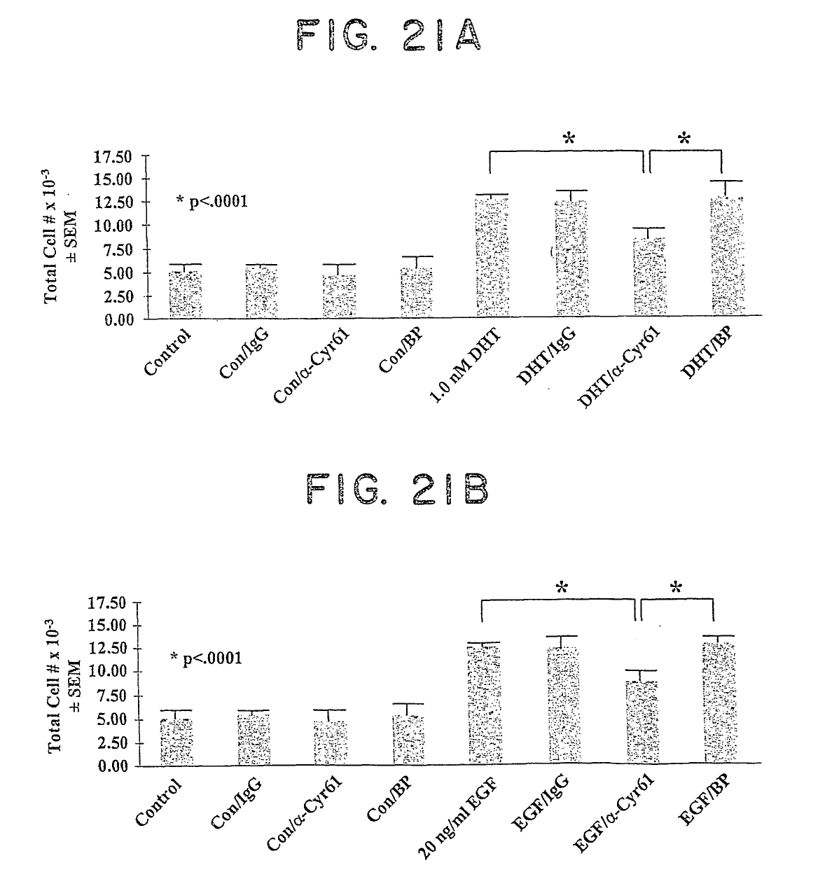

Results are shown in FIGS. 20-21. Initially, treatment of MDA-MB453 cells with 10 μg/ml of anti-Cyr61 reduced InM DHT and 20 ng/ml EGF induced DNA-synthesis by 47% and 43%, respectively (FIG. 20). Controls for the BrdU assay included co-treatment with 10 μg/ml non-immune IgG (IgG), which had no effect, and 10 μg/ml blocking peptide which completely reversed the neutralizing effects of anti-Cyr61. Therefore, Cyr61 appears to be necessary for initiation of cell cycle by effecting androgen and growth factor mediated entry into S-phase. Given that Cyr61 is necessary for DNA-synthesis, DHT and EGF-dependent MDA-MB-453 cell proliferation was monitored to directly determine the role of Cyr61 in cell growth. Anti-Cyr61 polyclonal antibodies (10 μg/ml) inhibited 1 nM DHT-dependent cell growth by 34% and EGF-dependent cell growth by 30% over a 10-day treatment period (FIGS. 21A and 21B). Moreover, the anti-proliferative effects were completely reversed upon co-treatment with a Cyr61 blocking peptide in which the neutralizing antibody was raised. The anti-proliferative effect of the neutralizing antibodies was not due to cytotoxicity since treated NMA-MB-453 cells excluded trypan blue (data not shown). Thus, Cyr61 is required for DHT and EGF-mediated cell proliferation of AR+ breast cancer cell in vitro.

### Example 10

### Cyr61 Neutralizing Antibody Effects on Progesterone Steroid Mediated Cell-Proliferation

Polyclonal antibodies were raised to a 65 a.a. peptide corresponding to the central domain of Cyr61 which was selected based on a lack of homology to other CCN family members and published effectiveness of the antibodies in neutralizing bFGF-mediated DNA synthesis in human microvascular endothelial cells. The antibodies were affinity purified and assayed for specificity by western blot analysis using E2-treated MCF-7 whole cell lysates. For cell proliferation assays, T47D cells were cultured in 2% charcoal stripped FBS media containing 1.0 nM R5020, 20 ng/ml EGF, a combination of R5020 and EGF, or a combination of R5020, EGF, and 100 nM RU486 for 5 days in the presence or absence of 10 g/ml of anti-Cyr61 neutralizing antibodies or non-immune IgG at 37° C. in 5% CO₂. Following steroid or growth factor treatment, monolayers were trypsinized, combined with cells in the culture supernatant, and counted in a Coulter Multisizer II counter (Coulter Corporation, Miami, Fla.).

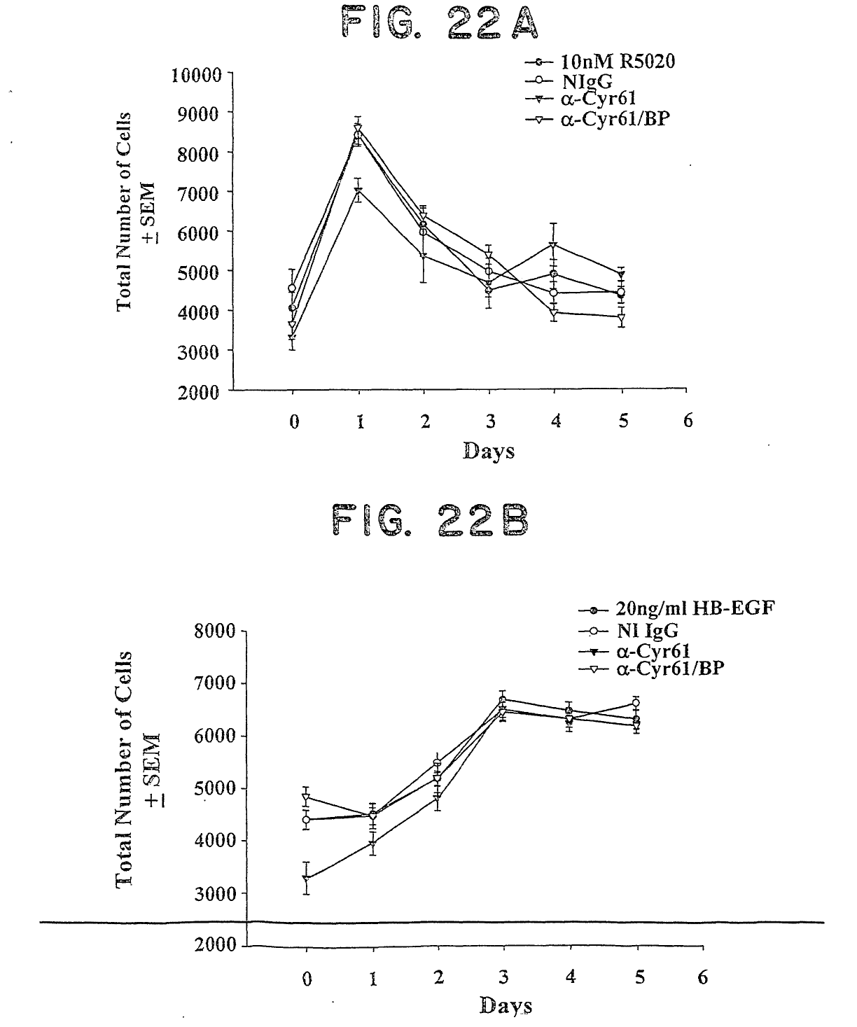

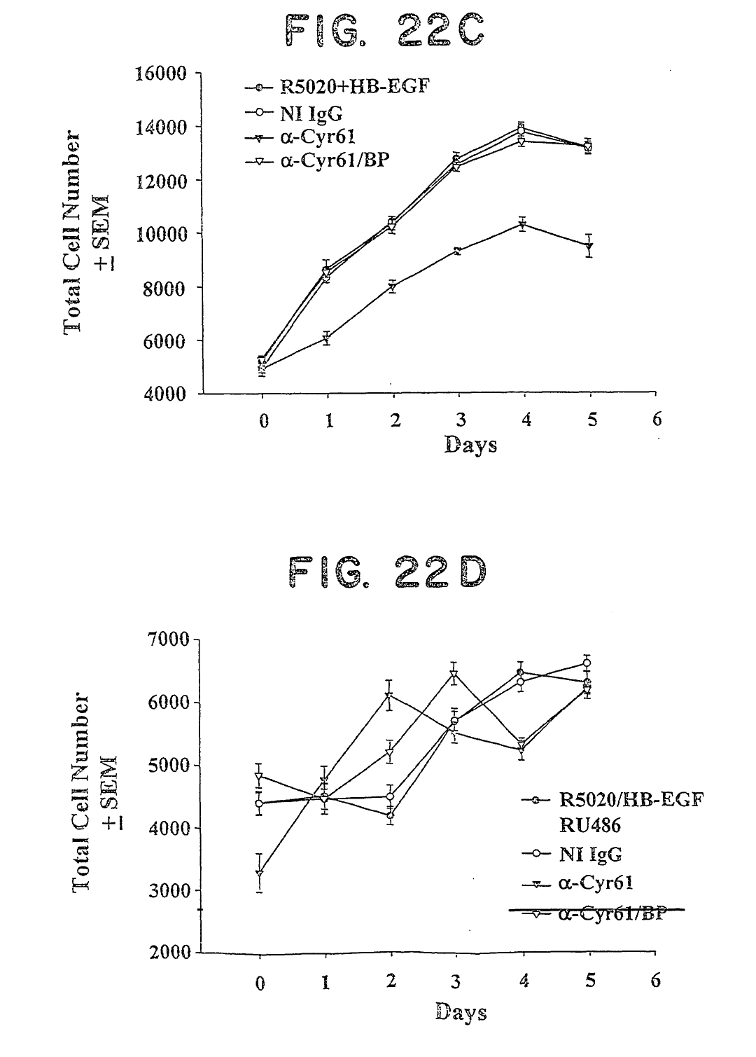

Results are shown in FIG. 22. A 30% decrease in cell growth when anti-Cyr61 neutralizing antibodies are present in the presence of the progestin R5020 and EGF (FIG. 22C) over a 5 day treatment period. More importantly, when RU486 is added to the combination of R5020 and EGF, the synergy between steroid and growth factor is lost suggesting that the induction of genes such as Cyr61 by progestin is required for the priming effect in T47D cells in vitro. This data demonstrates that Cyr61 is one mediator of progestin actions in priming breast cancer cells in vitro.

### Example 11

### Cyr61 Levels in Human Cancer Tumors Classified as PR+/EGFR+ and PR/EGFR+

Breast tumor biopsies and matched normal mammary tissue specimens were obtained from Clinomics Inc., (Pittsfield, Mass.) following informed patient consent and internal review board approval. Patients (n=20) were between the ages of 33-72 years and diagnosed with stage II invasive ductal carcinoma following histological examination. In addition, tumors were classified as PR+/ER−EGFR+ (n=10) or PR−/ER−EGFR+ (n=10) by immunohistochemical staining of formalin-fixed tumor biopsies using mono-specific anti-PR, anti-ER and anti-EGFR antibodies, respectively. Tissue specimens were immediately frozen in liquid nitrogen following surgery for protein extraction or fixed in 10% neutral-buffered formalin for in situ hybridization.

Tissue protein extracts were prepared from breast tumors and matched normal mammary tissue specimens by homogenization in 50 mM Tris, pH 8.0, 250 mM NaCl, 1.0% Nonidet P40, 1.0% Triton-X 100, 2.0% SDS, 0.5% deoxycholate, 1 mM EDTA, and protease inhibitor cocktail containing 10 μg/ml pepstatin, aprotinin, and leupeptin (SigmaAldrich). Protein extracts (20 μg) were subjected to SDS-polyacrylamide gel electrophoresis under reducing conditions in 10% bis-acrylamide and electrophoretically transferred to polyvinyl difluoride membrane (Immobilon-P, Biorad, Redding, Calif., USA). Membranes were blocked with 5% dry milk on TBS/0.1% Tween-20 (TBST), and incubated with anti-Cyr61 pAb (10 μg/ml). Primary antibody binding was detected using a Donkey anti-rabbit IgG antibody conjugated to horseradish peroxidase (HRP) and an enhanced chemiluminescence detection system (Amersham). All immunoblots were subsequently reprobed with 1 μg/ml of anti-pan-cytokeratin monoclonal antibodies (Sigma-Aldrich) to verify equivalent protein loading.

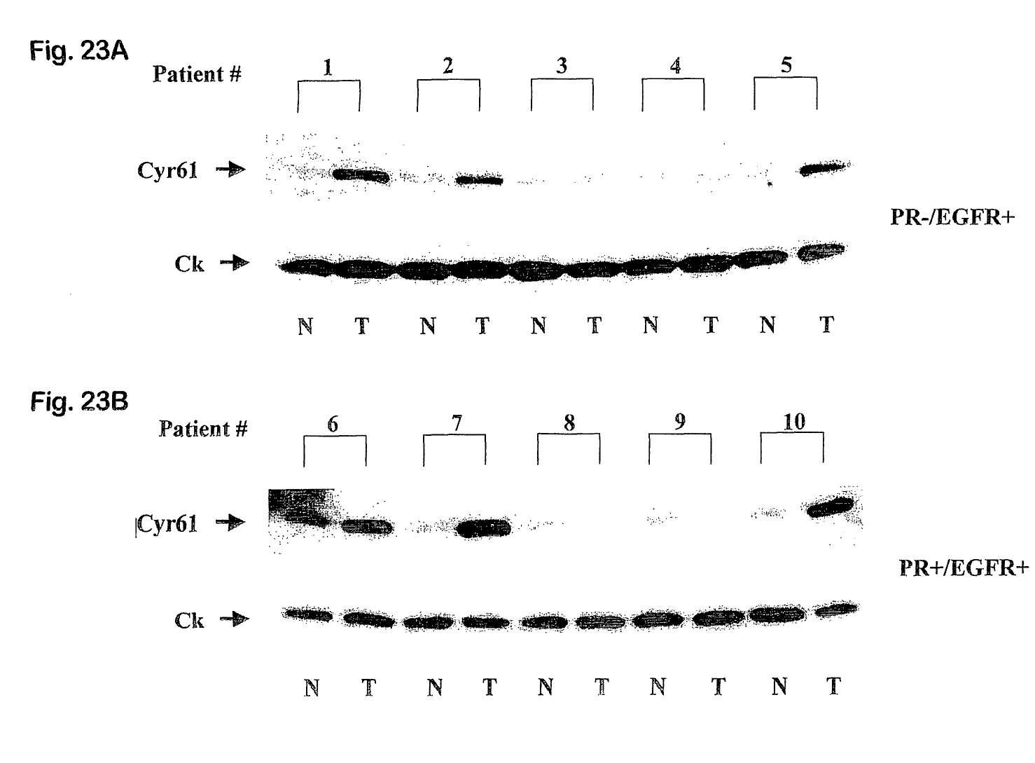

Results are shown in FIG. 23. These data suggest that the levels of Cyr61 protein correlates with progesterone receptor status in the absence of estrogen receptor suggesting that Cyr61 may be regulated by progesterone and its cognate receptor in vivo and in disease.

### Example 12

### Transgenic Animal Models

Mice that will selectively overexpress Cyr61 in mammary epithelium will be generated by utilizing the mouse mammary tumor virus (MMTV) system. Briefly, the human wild type Cyr61 cDNA will be cloned into an expression plasmid containing the full length MMTV-LTR (long terminal repeat), plus an SV40 intron and polyadenylation signals to generate MMTV-Cyr61. A linearized fragment from the MMTV-Cyr61 construct will be microinjected into fertilized C57/B1 6 mouse oocytes and reimplanted into C57/BL6 mice. Littermates will be genotyped for Cyr61 expression by isolating tail DNA and analyzed by southern blotting using a radiolabeled fragment of the Cyr61 cDNA according to previously described protocols (see Sambrook and Maniatis). At least 10 founder mice will be identified and mated to create the first generation of Cyr61 trangenic mice (F1 generation). If overexpression in the mouse mammary epithelium is accomplished than the predicted phenotype will be mammary hyperplasia and focal carcinoma formation.

The patents, applications, test methods, and publications mentioned herein are hereby incorporated by reference in their entirety.

Many variations of the present invention will suggest themselves to those skilled in the art in light of the above detailed description. All such obvious variations are within the full intended scope of the appended claims.

We claim:

1. An antibody which neutralizes Cyr61 activity.

2. An antibody as defined in claim 1, which is chimeric.

3. An antibody as defined in claim 1, which is humanly acceptable.

4. An antibody as defined in claim 1, which is conjugated to an anti-tumor agent.

5. An antibody as defined in claim 1, which is a monoclonal antibody.

6. An antibody which binds to amino acids 163-229 or to amino acids 371-382 of SEQ ID NO:2.

7. An antibody which binds to one or more ligands of a sex steroid receptor which regulates the promoter of the gene which encodes Cyr61.

8. An antibody as defined in claim 7, wherein said sex steroid receptor is selected from the group consisting of estrogen receptor, progesterone receptor, and androgen receptor.

9. An oligonucleotide which binds under high stringency conditions to a polynucleotide encoding Cyr61.

10. An oligonucleotide as defined in claim 9, wherein said oligonulceotide is non-naturally occurring.

11. A vector comprising an oligonucleotide as defined in claim 9.

12. A pharmaceutical composition comprising a therapeutically effective amount of at least one of (i) an antibody as defined in any of claims 1-8; (ii) an oligonucleotide as defined in any of claims 8-9; or (iii) a vector as defined in claim 11.

13. A method for preventing or inhibiting breast cancer cell proliferation, said method comprising administering to a subject a breast cancer cell proliferation blocking or reducing amount of a Cyr61 neutralizing antibody.

14. A method as defined in claim 13, wherein said neutralizing antibody blocks sex steroid induced synthesis of Cyr61 DNA and proliferation of breast cancer cells.

15. A method as defined in claim 13, wherein said neutralizing antibody blocks growth factor induced synthesis of Cyr61 DNA and proliferation of breast cancer cells.

16. A method as defined in claim 15, wherein said growth factor is selected from the group consisting of epidermal growth factor, heparin binding epidermal growth factor, and basic fibroblastic growth factor.

17. A method for diagnosing or staging breast cancer, said method comprising determining the level of Cyr61 in a breast cancer cell present in said breast cancer suspected of being positive for breast cancer and comparing said level to the level of Cyr61 in normal breast tissue, whereby an increase in the level of Cyr61 in said suspect tissue over the level of Cyr61 in said normal tissue indicates the presence of breast cancer in said suspect tissue.

18. A method as defined in claim 17, wherein said level of Cyr61 is determined by exposing said suspect and said normal tissues to a Cyr61 neutralizing antibody and comparing the amount of antibody bound by each tissue, wherein an increase in the level of bound antibody by said suspect tissue over the level of bound antibody by said normal tissue indicates the presence of breast cancer in said suspect tissue.

19. A method for diagnosing or staging breast cancer, said method comprising determining whether breast tissue suspected of being positive for breast cancer is (i) ER/Cyr61 positive, (ii) PR/Cyr61 positive, (iii) ER/PR/Cyr61 positive, (iv) AR/Cyr61 positive, or (v) PR/EGFR/Cyr61 positive; whereby the presence of (i), (ii), (iii), (iv), or (v) indicates a likelihood that said suspect tissue is cancerous.

20. A method of screening for a compound which inhibits or prevents breast cancer cell proliferation, said method comprising determining a first amount of Cyr61 expressed by breast cancer cells exposed to said compound, wherein said breast cancer cells overexpress Cyr61; and comparing said first amount of Cyr61 to a second amount of Cyr61 expressed by said breast cancer cells that have not been exposed to said compound; whereby said first amount being less than said second amount indicates that said compound may inhibit or prevent breast cancer cell proliferation.

21. A method of screening for a compound which inhibits or prevents breast cancer cell proliferation, said method comprising determining whether said compound inhibits the interaction of sex steroid response element of the Cyr61 promoter and a sex steroid receptor associated with the Cyr61 promoter.

22. A method of screening for a compound which inhibits or prevents breast cancer cell proliferation, said method comprising determining whether said compound binds with a sex steroid receptor which regulates the Cyr61 promoter.

23. A transgenic non-human animal comprising DNA which can be induced to overexpress Cyr61 in breast tissue.

24. The transgenic non-human animal as defined in claim 23, wherein the DNA is human.

25. A kit for diagnosing or staging breast cancer, said kit comprising an antibody as defined in any of claims 1-8.

26. A kit for diagnosing or staging breast cancer, said kit comprising an oligonucleotide as defined in claim 9 or 10.

27. A method for screening compounds that regulate Cyr61 mRNA transcription through a receptor, said method comprising detecting a difference in the level of Cyr61 mRNA in a population of cells sufficient to transcribe a detectable amount of mRNA encoding Cyr61 contacted with a test compound in comparison with the level of Cyr61 mRNA in such a population that is not contacted with said test compound.

28. An assay method for detecting the presence of breast cancer, said method comprising detecting the level of Cyr61 mRNA isolated from breast cancer tissue, in comparison with the level of Cyr61 mRNA isolated from normal mammary tissue; wherein an upregulation of Cyr61 mRNA compared to normal mammary tissue indicates the presence of breast cancer.

29. An assay method for detecting the presence of breast cancer, said method comprising detecting the level of Cyr61 protein isolated from breast cancer tissue, in comparison with the level of Cyr61 protein isolated from normal mammary tissue; wherein an upregulation of Cyr61 protein compared to normal mammary tissue indicates the presence of breast cancer.

30. A method for preventing or inhibiting breast cancer cell proliferation, said method comprising administering to a subject, an amount of a compound effective to inhibit the interaction of a sex steroid receptor with a sex steroid response element of the Cyr61 promoter.

31. A method as defined in claim 15, wherein said sex steroid is selected from the group consisting of an estrogenic compound, a progestational compound, and an androgenic compound.

32. A method as defined in claim 13 or 18, wherein said neutralizing antibody is an antibody of claims 1-8.

33. A method as defined in claim 22, 23 or 30, wherein said steroid receptor is selected from the group consisting of estrogen receptor, progesterone receptor, and androgen receptor.

34. The method as defined in claim 31, wherein said estrogenic compound is 17β-estradiol

35. An antibody which binds to an epitope of Cyr61.


SEQUENCE LISTING (6 sequences)

SEQ ID NO: 1 - DNA, 2016 bp, Homo sapiens
ggcacgaggagcagcgcccgcgccctccgcgccttctccgccgggacctcgagcgaaagacgcccgcccgccgcccagccctcgcctccctgcccaccgggcccaccgcgccgccaccccgaccccgctgcgcacggcctgtccgctgcacaccagcttgttggcgtcttcgtcgccgcgctcgccccgggctactcctgcgcgccacaatgagctcccgcatcgccagggcgctcgccttagtcgtcacccttctccacttgaccaggctggcgctctccacctgccccgctgcctgccactgccccctggaggcgcccaagtgcgcgccgggagtcgggctggtccgggacggctgcggctgctgtaaggtctgcgccaagcagctcaacgaggactgcagcaaaacgcagccctgcgaccacaccaaggggctggaatgcaacttcggcgccagctccaccgctctgaaggggatctgcagagctcagtcagagggcagaccctgtgaatataactccagaatctaccaaaacggggaaagtttccagcccaactgtaaacatcagtgcacatgtattgatggcgccgtgggctgcattcctctgtgtccccaagaactatctctccccaacttgggctgtcccaaccctcggctggtcaaagttaccgggcagtgctgcgaggagtgggtctgtgacgaggatagtatcaaggaccccatggaggaccaggacggcctccttggcaaggagctgggattcgatgcctccgaggtggagttgacgagaaacaatgaattgattgcagttggaaaaggcagctcactgaagcggctccctgtttttggaatggagcctcgcatcctatacaaccctttacaaggccagaaatgtattgttcaaacaacttcatggtcccagtgctcaaagacctgtggaactggtatctccacacgagttaccaatgacaaccctgagtgccgccttgtgaaagaaacccggatttgtgaggtgcggccttgtggacagccagtgtacagcagcctgaaaaagggcaagaaatgcagcaagaccaagaaatcccccgaaccagtcaggtttacttacgctggatgtttgagtgtgaagaaataccggcccaagtactgcggttcctgcgtggacggccgatgctgcacgccccagctgaccaggactgtgaagatgcggttccgctgcgaagatggggagacattttccaagaacgtcatgatgatccagtcctgcaaatgcaactacaactgcccgcatgccaatgaagcagcgtttcccttctacaggctgttcaatgacattcacaaatttagggactaaatgctacctgggtttccagggcacacctagacaaacaagggagaagagtgtcagaatcagaatcatggagaaaatgggcgggggtggtgtgggtgatgggactcattgtagaaaggaagccttgctcattcttgaggagcattaaggtatttcgaaactgccaagggtgctggtgcggatggacactaatgcagccacgattggagaatactttgcttcatagtattggagcacatgttactgcttcattttggagcttgtggagttgatgactttctgttttctgtttgtaaattatttgctaagcatattttctctaggcttttttccttttggggttctacagtcgtaaaagagataataagattagttggacagtttaaagcttttattcgtcctttgacaaaagtaaatgggagggcattccatcccttcctgaagggggacactccatgagtgtctgtgagaggcagctatctgcactctaaactgcaaacagaaatcaggtgttttaagactgaatgttttatttatcaaaatgtagcttttggggagggaggggaaatgtaatactggaataatttgtaaatgattttaattttatattcagtgaaaagattttatttatggaattaaccatttaataaagaaatatttacct

SEQ ID NO: 2 - protein, 381 aa, Homo sapiens [sequence omitted]

SEQ ID NO: 3 - DNA, 13 bp, Artificial Sequence
  consensus sequence for estrogen response element
ggtcannntgacc

SEQ ID NO: 4 - DNA, 15 bp, Artificial Sequence
  consensus sequence for progesterone receptor/androgen receptor element
tgtacannntgttct

SEQ ID NO: 5 - DNA, 1418 bp, Homo sapiens
  CDS (124)..(1266)
gggcgggcccaccgcgacaccgcgccgccaccccgaccccgctgcgcacggcctgtccgctgcacaccagcttgttggcgtcttcgtcgccgcgctcgccccgggctactcctgcgcgccacaatgagctcccgcatcgccagggcgctcgccttagtcgtcacccttctccacttgaccaggctggcgctctccacctgccccgctgcctgccactgccccctggaggcgcccaagtgcgcgccgggagtcgggctggtccgggacggctgcggctgctgtaaggtctgcgccaagcagctcaacgaggactgcagcaaaacgcagccctgcgaccacaccaaggggctggaatgcaacttcggcgccagctccaccgctctgaaggggatctgcagagctcagtcagagggcagaccctgtgaatataactccagaatctaccaaaacggggaaagtttccagcccaactgtcaacatcagtgcacatgtattgatggcgccgtgggctgcattcctctgtgtccccaagaactatctctccccaacttgggctgtcccaaccctcggctggtcaaagttaccgggcagtgctgcgaggagtgggtctgtgacgaggatagtatcaaggaccccatggaggaccaggacggcctccttggcaaggagctgggattcgatgcctccgaggtggagttgacgagaaacaatgaattgattgcagttggaaaaggcagatcactgaagcggctccctgtttttggaatggagcctcgcatcctatacaaccctttacaaggccagaaatgtattgttcaaacaacttcatggtcccagtgctcaaagacctgtggaactggtatctccacacgagttaccaatgacaaccctgagtgccgccttgtgaaagaaacccggatttgtgaggtgcggccttgtggacagccagtgtacagcagcctgaaaaagggcaagaaatgcagcaagaccaagaaatcccccgaaccagtcaggtttacttacgctggatgtttgagtgtgaagaaataccggcccaagtactgcggttcctgcgtggacggccgatgctgcacgccccagctgaccaggactgtgaagatgcggttccgctgcgaagatggggagacattttccaagaacgtcatgatgatccagtcctgcaaatgcaactacaactgcccgcatgccaatgaagcagcgtttcccttctacaggctgttcaatgacattcacaaatttagggactaaatgctacctgggtttccagggcacacctagacaaacaagggagaagagtgtcagaatcagaatcatggagaaaatgggcgggggtggtgtgggtgatgggactcattgtagaaaggaagccttctcattcttgaggagcattaaggtat

SEQ ID NO: 6 - protein, 381 aa, Homo sapiens
MSSRIARALALVVTLLHLTRLALSTCPAACHCPLEAPKCAPGVGLVRDGCGCCKVCAKQLNEDCSKTQPCDHTKGLECNFGASSTALKGICRAQSEGRPCEYNSRIYQNGESFQPNCQHQCTCIDGAVGCIPLCPQELSLPNLGCPNPRLVKVTGQCCEEWVCDEDSIKDPMEDQDGLLGKELGFDASEVELTRNNELIAVGKGRSLKRLPVFGMEPRILYNPLQGQKCIVQTTSWSQCSKTCGTGISTRVTNDNPECRLVKETRICEVRPCGQPVYSSLKKGKKCSKTKKSPEPVRFTYAGCLSVKKYRPKYCGSCVDGRCCTPQLTRTVKMRFRCEDGETFSKNVMMIQSCKCNYNCPHANEAAFPFYRLFNDIHKFRD